SEQUENCE LISTING (37 sequences)

SEQ ID NO: 1 - DNA, 13 bp, Artificial Sequence
  EcoKI site
  N (4)..(9): N is any nucleotide
  misc_feature (4)..(9): n is a, c, g, or t
aacnnnnnngtgc

SEQ ID NO: 2 - DNA, 13 bp, Artificial Sequence
  EcoKI site
  N (5)..(10): N is any nucleotide
  misc_feature (5)..(10): n is a, c, g, or t
gcacnnnnnngtt

SEQ ID NO: 3 - DNA, 12 bp, Artificial Sequence
  BcgI site
  N (6)..(9): N is any nucleotide
  misc_feature (6)..(9): n is a, c, g, or t
cgatcnnnntgc

SEQ ID NO: 4 - DNA, 10 bp, Artificial Sequence
  BsaBI site
  N (5)..(7): N is any nucleotide
  misc_feature (5)..(7): n is a, c, g, or t
gatcnnnatc

SEQ ID NO: 5 - DNA, 10 bp, Artificial Sequence
  Nt.AlwI site
  N (6)..(10): N is any nucleotide
  misc_feature (6)..(10): n is a, c, g, or t
ggatcnnnnn

SEQ ID NO: 6 - DNA, 10 bp, Artificial Sequence
  AlwNI site
  N (4)..(5): N is any nucleotide
  misc_feature (4)..(5): n is a, c, g, or t
cagnncctgg

SEQ ID NO: 7 - DNA, 11 bp, Artificial Sequence
  BslI site
  N (6)..(9): N is any nucleotide
  misc_feature (6)..(9): n is a, c, g, or t
ccwggnnnngg

SEQ ID NO: 8 - DNA, 12 bp, Artificial Sequence
  BstXI site
  N (6)..(9): N is any nucleotide
  misc_feature (6)..(9): n is a, c, g, or t
ccaggnnnntgg

SEQ ID NO: 9 - DNA, 11 bp, Artificial Sequence
  PflMI site
  N (6)..(8): N is any nucleotide
  misc_feature (6)..(8): n is a, c, g, or t
ccaggnnntgg

SEQ ID NO: 10 - DNA, 13 bp, Artificial Sequence
  SfiI site
  W (5)..(5): W is A or T
  N (8)..(9): N is any nucleotide
  misc_feature (8)..(9): n is a, c, g, or t
ggccwggnnggcc

SEQ ID NO: 11 - DNA, 16 bp, Artificial Sequence
  SfiI site
  N (5)..(9): N is any nucleotide
  misc_feature (5)..(9): n is a, c, g, or t
  W (14)..(14): W is A or T
ggccnnnnnggccwgg

SEQ ID NO: 12 - DNA, 25 bp, Artificial Sequence
  AbaSI site
  ghm (1)..(1): glucosylhydroxymethylcytosine modification
  N (2)..(24): N is any nucleotide
  misc_feature (2)..(24): n is a, c, g, or t
cnnnnnnnnnnnnnnnnnnnnnnng

SEQ ID NO: 13 - DNA, 24 bp, Artificial Sequence
  AbaSI site
  ghm (1)..(1): glucosylhydroxymethylcytosine
  N (2)..(23): N is any nucleotide
  misc_feature (2)..(23): n is a, c, g, or t
cnnnnnnnnnnnnnnnnnnnnnng

SEQ ID NO: 14 - DNA, 23 bp, Artificial Sequence
  AbaSI site
  ghm (1)..(1): glucosylhydroxymethylcytosine modification
  N (2)..(22): N is any nucleotide
  misc_feature (2)..(22): n is a, c, g, or t
cnnnnnnnnnnnnnnnnnnnnng

SEQ ID NO: 15 - DNA, 22 bp, Artificial Sequence
  AbaSI site
  ghm (1)..(1): glucosylhydroxymethylcytosine modification
  N (2)..(21): N is any nucleotide
  misc_feature (2)..(21): n is a, c, g, or t
cnnnnnnnnnnnnnnnnnnnng

SEQ ID NO: 16 - DNA, 25 bp, Artificial Sequence
  AbaSI site
  N (2)..(24): N is any nucleotide
  misc_feature (2)..(24): n is a, c, g, or t
  * (25)..(25): 5-glucosylhydroxymethylcytosine, 5-hydroxymethylcytosine, 5-methylcytosine or cytosine
gnnnnnnnnnnnnnnnnnnnnnnnc

SEQ ID NO: 17 - DNA, 24 bp, Artificial Sequence
  AbaSI site
  N (2)..(23): N is any nucleotide
  misc_feature (2)..(23): n is a, c, g, or t
  * (24)..(24): 5-glucosylhydroxymethylcytosine, 5-hydroxymethylcytosine, 5-methylcytosine or cytosine
gnnnnnnnnnnnnnnnnnnnnnnc

SEQ ID NO: 18 - DNA, 23 bp, Artificial Sequence
  AbaSI site
  N (2)..(22): N is any nucleotide
  misc_feature (2)..(22): n is a, c, g, or t
  * (23)..(23): 5-glucosylhydroxymethylcytosine, 5-hydroxymethylcytosine, 5-methylcytosine or cytosine
gnnnnnnnnnnnnnnnnnnnnnc

SEQ ID NO: 19 - DNA, 22 bp, Artificial Sequence
  AbaSI site
  N (2)..(21): N is any nucleotide
  misc_feature (2)..(21): n is a, c, g, or t
  * (22)..(22): 5-glucosylhydroxymethylcytosine, 5-hydroxymethylcytosine, 5-methylcytosine or cytosine
gnnnnnnnnnnnnnnnnnnnnc

SEQ ID NO: 20 - DNA, 14 bp, Artificial Sequence
  FspEI site
  mC (2)..(2): 5-methylcytosine or 5-hydroxymethylcytosine
  N (2)..(14): N is any nucleotide
  misc_feature (3)..(14): n is a, c, g, or t
ccnnnnnnnnnnnn

SEQ ID NO: 21 - DNA, 18 bp, Artificial Sequence
  FspEI site
  N (3)..(18): N is any nucleotide
  misc_feature (3)..(18): n is a, c, g, or t
ggnnnnnnnnnnnnnnnn

SEQ ID NO: 22 - DNA, 14 bp, Artificial Sequence
  LpnPI site
  mC (2)..(2): 5-methylcytosine or 5-hydroxymethylcytosine
  D (3)..(3): D is A, G, or T
  N (5)..(14): N is any nucleotide
  misc_feature (5)..(14): n is a, c, g, or t
ccdgnnnnnnnnnn

SEQ ID NO: 23 - DNA, 18 bp, Artificial Sequence
  LpnPI site
  H (3)..(3): H is A, C or T
  N (5)..(18): N is any nucleotide
  misc_feature (5)..(18): n is a, c, g, or t
gghcnnnnnnnnnnnnnn

SEQ ID NO: 24 - DNA, 13 bp, Artificial Sequence
  MspJI site
  mC (1)..(1): 5-methylcytosine or 5-hydroxymethylcytosine
  N (2)..(3): N is any nucleotide
  misc_feature (2)..(3): n is a, c, g, or t
  R (4)..(4): R is A or G
  N (5)..(13): N is any nucleotide
  misc_feature (5)..(13): n is a, c, g, or t
cnnrnnnnnnnnn

SEQ ID NO: 25 - DNA, 17 bp, Artificial Sequence
  MspJI site
  N (2)..(3): N is any nucleotide
  misc_feature (2)..(3): n is a, c, g, or t
  Y (4)..(4): Y is C or T
  N (5)..(17): N is any nucleotide
  misc_feature (5)..(17): n is a, c, g, or t
gnnynnnnnnnnnnnnn

SEQ ID NO: 26 - DNA, 22 bp, Artificial Sequence
  sequence encoding crRNA
gttttagagctatgctgttttg

SEQ ID NO: 27 - DNA, 86 bp, Artificial Sequence
  sequence encoding tracrRNA
ggaaccattcaaaacagcatagcaagttaaaataaggctagtccgttatcaacttgaaaaagtggcaccgagtcggtgcttttttt

SEQ ID NO: 28 - DNA, 83 bp, Artificial Sequence
  gNA sequence
gttttagagctagaaatagcaagttaaaataaggctagtccgttatcaacttgaaaaagtggcaccgagtcggtgcttttttt

SEQ ID NO: 29 - DNA, 83 bp, Artificial Sequence
  gNA sequence
aaaaaaagcaccgactcggtgccactttttcaagttgataacggactagccttattttaacttgctatttctagctctaaaac

SEQ ID NO: 30 - RNA, 83 nt, Artificial Sequence
  gRNA sequence
guuuuagagcuagaaauagcaaguuaaaauaaggcuaguccguuaucaacuugaaaaaguggcaccgagucggugcuuuuuuu

SEQ ID NO: 31 - DNA, 94 bp, Artificial Sequence
  gNA sequence
gttttagagctatgctggaaacagcatagcaagttaaaataaggctagtccgttatcaacttgaaaaagtggcaccgagtcggtgctttttttc

SEQ ID NO: 32 - DNA, 94 bp, Artificial Sequence
  gNA sequence
gaaaaaaagcaccgactcggtgccactttttcaagttgataacggactagccttattttaacttgctatgctgtttccagcatagctctaaaac

SEQ ID NO: 33 - RNA, 94 nt, Artificial Sequence
  gRNA sequence
guuuuagagcuaugcuggaaacagcauagcaaguuaaaauaaggcuaguccguuaucaacuugaaaaaguggcaccgagucggugcuuuuuuuc

SEQ ID NO: 34 - RNA, 19 nt, Artificial Sequence
  gRNA sequence
aauuucuacuguuguagau

SEQ ID NO: 35 - DNA, 19 bp, Artificial Sequence
  gNA sequence
aatttctactgttgtagat

SEQ ID NO: 36 - RNA, 22 nt, Artificial Sequence
  gRNA sequence
guuuuagagcuaugcuguuuug

SEQ ID NO: 37 - RNA, 86 nt, Artificial Sequence
  gRNA sequence
ggaaccauucaaaacagcauagcaaguuaaaauaaggcuaguccguuaucaacuugaaaaaguggcaccgagucggugcuuuuuuu

(19) United States
(12) Patent Application Publication
(10) Pub. No.: US 2022/0186290 A1
(43) Pub. Date: Jun. 16, 2022

(54) COMPOSITIONS AND METHODS FOR NUCLEOTIDE MODIFICATION-BASED DEPLETION

(71) Applicant: ARC BIO, LLC, Cambridge, MA (US)

(72) Inventor: Stephane B. GOURGUECHON, San Mateo, CA (US)

(21) Appl. No.: 17/602,577

(22) PCT Filed: Apr. 8, 2020
(86) PCT No.: PCT/US2020/027293

§ 371 Date: Oct. 8, 2021

Related U.S. Application Data
(60) Provisional application No. 62/831,302, filed on Apr. 9, 2019.

(51) Int. Cl. C12Q 1/6806; C12N 15/11; C12N 9/22

(52) U.S. Cl. CPC C12Q 1/6806; C12N 15/111; C12N 2330/31; C12N 2310/20; C12N 9/22

(57) ABSTRACT

Provided herein are compositions and methods for enriching a sample for nucleic acids of interest relative to nucleic acids targeted for depletion, comprising using differences in nucleotide modification between the nucleic acids of interest and the nucleic acids targeted for depletion.

### CROSS REFERENCE TO RELATED APPLICATIONS

This application claims priority to and benefit of U.S. Provisional Application No. 62,831,302, filed Apr. 9, 2019, the contents of which are hereby incorporated by reference in their entirety.

### INCORPORATION OF THE SEQUENCE LISTING

The contents of the text file submitted electronically herewith are incorporated herein by reference in their entirety: a computer readable format copy of the Sequence Listing (filename: ARCB_01301WO_SeqList, date recorded: Apr. 6, 2020, file size: 13 KB).

### BACKGROUND

Human clinical DNA samples and sample libraries such as cDNA libraries derived from RNA contain sequences that have little informative value and increase the cost of sequencing. While methods have been developed to deplete these unwanted sequences (e.g., via hybridization capture) and enrich for sequences of interest, these methods are often time-consuming and can be expensive. There thus exists a need in the art for methods to deplete unwanted sequences from libraries. The invention provides methods for depleting sequences from libraries and enriching for desirable sequences using differences in nucleotide modification between sequences of interest and sequences targeted for depletion.

### SUMMARY

The disclosure provides methods of enriching a sample for nucleic acids of interest relative to nucleic acids targeted for depletion by about at least about 2-fold, comprising using differences in nucleotide modification between the nucleic acids of interest and the nucleic acids targeted for depletion.

The disclosure provides methods of enriching a sample for nucleic acids of interest relative to nucleic acids targeted for depletion by about at least about 2-fold, comprising using differences in nucleotide modification between the nucleic acids of interest and the nucleic acids targeted for depletion, and not comprising size selection or modification-sensitive targeted binding.

The disclosure provides methods of enriching a sample for nucleic acids of interest relative to nucleic acids targeted for depletion by about at least about 2-fold, comprising using differences in nucleotide modification between the nucleic acids of interest and the nucleic acids targeted for depletion to ligate adapters to the nucleic acids of interest and not to the nucleic acids targeted for depletion.

The disclosure provides methods of enriching a sample for nucleic acids of interest comprising: (a) providing a sample comprising nucleic acids of interest and nucleic acids targeted for depletion, wherein at least a subset of the nucleic acids of interest or a subset of the nucleic acids targeted for depletion comprise a plurality of first recognition sites for a first modification-sensitive restriction enzyme; (b) terminally dephosphorylating a plurality of the nucleic acids in the sample; (c) contacting the sample from (b) with the first modification-sensitive restriction enzyme under conditions that allow for cleavage of at least some of the first modification-sensitive restriction sites in the nucleic acids in the sample; and (d) contacting the sample from (c) with adapters under conditions that allow for the ligation of the adapters to a 5′ and 3′ end of a plurality of the nucleic acids of interest; thereby generating a sample enriched for nucleic acids of interest that are adapter-ligated on their 5′ and 3′ ends.

In some embodiments of the methods of disclosure, both the nucleic acids of interest and the nucleic acids targeted for depletion each comprise a plurality of first recognition sites for the first modification-sensitive restriction enzyme. In some embodiments, a frequency of nucleotide modification within or adjacent to the plurality of first recognitions sites is not the same in nucleic acids of interest as in the nucleic acids targeted for depletion.

In some embodiments of the methods of the disclosure, activity of the first modification-sensitive restriction enzyme is blocked by modification of a nucleotide within or adjacent to its cognate recognition site. In some embodiments, the plurality of first recognition sites in the nucleic acids targeted for depletion are modified more frequently than the plurality of first recognition sites in the nucleic acids of interest.

In some embodiments of the methods of the disclosure, the first modification-sensitive restriction enzyme is active at a recognition site comprising at least one modified nucleotide and is not active at a recognition site that does not comprise at least one modified nucleotide. In some embodiments, the plurality of first recognition sites in the nucleic acids targeted for depletion are modified more frequently than the plurality of first recognition sites in the nucleic acids of interest.

In some embodiments of the methods of the disclosure, the methods further comprise, prior to step (d), contacting the sample from (c) with an exonuclease under conditions that allow for the successive removal of nucleotides from a phosphorylated end of a nucleic acid.

In some embodiments of the methods of the disclosure, the methods further comprise (e) contacting the adapter-ligated nucleic acids from (d) with a second modification-sensitive restriction enzyme under conditions that allow the second modification-sensitive restriction enzyme to cut a second recognition site, wherein at least a subset of the nucleic acids targeted for depletion comprise a plurality of second recognition sites for a second modification-sensitive restriction enzyme, and wherein the second modification-sensitive restriction enzyme targets recognition sites comprising at least one modified nucleotide and does not target recognition sites that do not comprise at least one modified nucleotide, thereby generating a collection of nucleic acids targeted for depletion that are adapter-ligated on one end and a collection of nucleic acids of interest that are adapter-ligated on both ends.

In some embodiments of the methods of the disclosure, the methods further comprise contacting the sample after step (d) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends. In some embodiments, the method comprises contacting the sample with at least 10² unique nucleic acid-guided nuclease-gNA complexes, at least 10³ unique nucleic acid-guided nuclease-gNA complexes, 10⁴ unique nucleic acid-guided nuclease-gNA complexes or 10⁵ unique nucleic acid-guided nuclease-gNA complexes. In some embodiments, the nucleic acid-guided nuclease is Cas9, Cpf1 or a combination thereof.

The disclosure provides methods of enriching a sample for nucleic acids of interest comprising: (a) providing a sample comprising nucleic acids of interest and nucleic acids targeted for depletion, wherein at least a subset of the nucleic acids targeted for depletion comprise a plurality of recognition sites for a modification-sensitive restriction enzyme; (b) terminally dephosphorylating a plurality of the nucleic acids in the sample; (c) contacting the sample from (b) with the modification-sensitive restriction enzyme under conditions that allow for the cleavage of the modification-sensitive restriction sites in the nucleic acids in the sample, thereby generating nucleic acids with exposed terminal phosphates; and (d) contacting the sample with an exonuclease under conditions that allow for the successive removal of nucleotides from a phosphorylated end of a nucleic acid; thereby generating a sample enriched for nucleic acids of interest.

In some embodiments of the methods of the disclosure, the nucleic acids of interest and the nucleic acids targeted for depletion each comprise a plurality of recognition sites for the modification-sensitive restriction enzyme. In some embodiments, the plurality of recognition sites in the nucleic acids targeted for depletion are modified more frequently than the plurality of recognition sites in the nucleic acids of interest.

In some embodiments of the methods of the disclosure, the methods further comprise (e) contacting the sample from (d) with adapters under conditions that allow for the ligation of the adapters to a 5′ and 3′ end of a plurality of the nucleic acids of interest; thereby generating a sample enriched for nucleic acids of interest that are adapter-ligated on their 5′ and 3′ ends.

In some embodiments of the methods of the disclosure, the methods further comprise contacting the sample after step (d) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends. In some embodiments, the method comprises contacting the sample with at least 10² unique nucleic acid-guided nuclease-gNA complexes, at least 10³ unique nucleic acid-guided nuclease-gNA complexes, 10⁴ unique nucleic acid-guided nuclease-gNA complexes or 10⁵ unique nucleic acid-guided nuclease-gNA complexes. In some embodiments, the nucleic acid-guided nuclease is Cas9, Cpf1 or a combination thereof.

The disclosure provides methods of enriching a sample for nucleic acids of interest comprising: (a) providing a sample comprising nucleic acids of interest and nucleic acids targeted for depletion, wherein at least a subset of the nucleic acids targeted for depletion comprise a plurality of recognition sites for a modification-sensitive restriction enzyme; (b) contacting the sample with adapters under conditions that allow for the ligation of the adapters to a 5′ and 3′ end of a plurality of the nucleic acids in the sample; and (c) contacting the sample from (b) with the modification-sensitive restriction enzyme under conditions that allow for the cleavage of the modification-sensitive restriction sites in the nucleic acids in the sample; thereby generating a sample enriched for nucleic acids of interest that are adapter-ligated on their 5′ and 3′ ends.

In some embodiments of the methods of the disclosure, both the nucleic acids of interest and the nucleic acids targeted for depletion each comprise a plurality of recognition sites for the modification-sensitive restriction enzyme. In some embodiments, the plurality of recognition sites in the nucleic acids targeted for depletion are modified more frequently than the plurality of recognition sites in the nucleic acids of interest.

In some embodiments of the methods of the disclosure, the methods further comprise contacting the sample after step (d) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends. In some embodiments, the methods comprise contacting the sample with at least 10² unique nucleic acid-guided nuclease-gNA complexes, at least 10³ unique nucleic acid-guided nuclease-gNA complexes, 10⁴ unique nucleic acid-guided nuclease-gNA complexes or 10⁵ unique nucleic acid-guided nuclease-gNA complexes. In some embodiments, the nucleic acid-guided nuclease is Cas9, Cpf1 or a combination thereof.

The disclosure provides methods of enriching a sample for nucleic acids of interest comprising: (a) providing a sample comprising nucleic acids of interest and nucleic acids targeted for depletion, wherein at least a subset of the nucleic acids of interest or a subset of the nucleic acids targeted for depletion comprise a plurality of first recognition sites for a first modification-sensitive restriction enzyme, and wherein activity of the first modification-sensitive restriction enzyme is blocked by modification of a nucleotide within or adjacent to its cognate recognition site; (b) terminally dephosphorylating a plurality of the nucleic acids in the sample; (c) contacting the sample from (b) with the first modification-sensitive restriction enzyme under conditions that allow for cleavage of at least some of the first modification-sensitive restriction sites in the nucleic acids in the sample; and (d) contacting the sample from (c) with adapters under conditions that allow for the ligation of the adapters to a 5′ and 3′ end of a plurality of the nucleic acids of interest; thereby generating a sample enriched for nucleic acids of interest that are adapter-ligated on their 5′ and 3′ ends.

In some embodiments of the methods of the disclosure, both the nucleic acids of interest and the nucleic acids targeted for depletion each comprise a plurality of first recognition sites for the first modification-sensitive restriction enzyme. In some embodiments, a frequency of nucleotide modification within or adjacent to the plurality of first recognitions sites is not the same in nucleic acids of interest as in the nucleic acids targeted for depletion. In some embodiments, the plurality of first recognition sites in the nucleic acids targeted for depletion are modified more frequently than the plurality of first recognition sites in the nucleic acids of interest.

In some embodiments of the methods of the disclosure, the methods further comprise amplifying, sequencing or cloning the nucleic acids of interest that are adapter-ligated on their 5′ and 3′ ends using the adapters.

In some embodiments, the nucleotide modification comprises adenine modification or cytosine modification. In some embodiments, the adenine modification comprises adenine methylation. In some embodiments, the adenine methylation comprises Dam methylation or EcoKI methylation. In some embodiments, the cytosine modification comprises 5-methylcytosine, 5-hydroxymethlcytosine, 5-formylcytosine, 5-carboxylcytosine, 5-glucosylhydroxymethylcytosine or 3-methylcytosine. In some embodiments, the cytosine modification comprises cytosine methylation. In some embodiments, cytosine methylation comprises CpG methylation, CpA methylation, CpT methylation, CpC methylation or a combination thereof. In some embodiments, the cytosine methylation comprises Dcm methylation, DNMT1 methylation, DNMT3A methylation or DNMT3B methylation.

In some embodiments, the nucleic acids targeted for depletion comprise host nucleic acids and the nucleic acids of interest comprise non-host nucleic acids.

### BRIEF DESCRIPTION OF THE DRAWINGS

FIG. 1 is a diagram illustrating an exemplary method of the disclosure. Nucleic acids in the sample are dephosphorylated, and then digested with a restriction enzyme that is blocked by the presence of modifications at the restriction enzyme recognition site. The exposed phosphates from the resulting digestion are then used to ligate adapters to the nucleic acids of interest.

FIG. 2 is a diagram illustrating an exemplary method of the disclosure. Nucleic acids in the sample are dephosphorylated, and then digested with a restriction enzyme that recognizes a restriction enzyme site comprising one or more modified nucleotides. Cut nucleic acids are then digested with an exonuclease that uses the exposed terminal phosphates, and adapters are ligated to the remaining nucleic acids of interest.

FIG. 3 is a diagram illustrating an exemplary method of the disclosure. Nucleic acids in the sample are adapter ligated, and then digested with a restriction enzyme that recognizes a restriction enzyme site comprising one or more modified nucleotides, resulting in nucleic acids of interest that are adapter ligated on both ends.

FIG. 4 is a diagram illustrating an exemplary method of the disclosure. Nucleic acids in the sample are adapter ligated, and then cleaved with a nucleic acid-guided nuclease that cleaves the nucleic acids targeted for depletion, resulting in nucleic acids of interest that are adapter ligated on both ends. This method can be used in conjunction with the nucleotide modification based methods of the disclosure.

### DETAILED DESCRIPTION

Epigenetic nucleotide modifications within the genome vary between species. For example, the frequency and type of nucleotide modification differs between vertebrates and bacteria, fungi or viruses. Furthermore, modifications such as methylation also occur more frequently in some genomes, such as the human genome, at transcriptionally active sites (e.g. genes and/or promoters of genes), and less frequently at other sites in the genome (e.g. repetitive regions). Some restriction enzymes are sensitive to nucleotide modification at or adjacent to their cognate recognition sites. It possible to exploit differences in nucleotide modification between sequences to enrich a sample for nucleic acids of interest using modification-sensitive restriction enzymes.

The disclosure provides methods of enriching a sample for nucleic acids of interest relative to nucleic acids targeted for depletion, comprising using differences in nucleotide modification frequency between the nucleic acids of interest and nucleic acids targeted for depletion. The methods of the disclosure allow for reductions in library complexity, and enrichment for sequences that can be used in a variety of downstream applications, including but not limited to, PCR amplification, cloning, high throughput sequencing, identification of rare sequences in a mixed population, and quantification of sequences within a library. In some embodiments, the sample is enriched for nucleic acids of interest by at least about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 40 fold, about 50 fold, about 100 fold, 200 fold about 500 fold or about 1000 fold. In some embodiments, the sample is enriched for nucleic acids of interest by at least about 2 fold. In some embodiments, the sample is enriched for nucleic acids of interest by at least about 3 fold. In some embodiments, the sample is enriched for nucleic acids of interest by about 2 fold to about 3 fold. In some embodiments, the sample is enriched for nucleic acids of interest by at least about 12-fold. In some embodiments, the sample is enriched for nucleic acids of interest by at least about 15-fold. In some embodiments, the sample is depleted of nucleic acids targeted for depletion by at least about 50% to about 70%. In some embodiments, the sample is depleted of nucleic acids targeted for depletion by at least about 95%.

The disclose provides methods of enriching a sample for nucleic acids of interest comprising: (a) providing a sample comprising nucleic acids of interest and nucleic acids targeted for depletion, wherein at least a subset of the nucleic acids of interest or a subset of the nucleic acids targeted for depletion comprise a plurality of first recognition sites for a first modification-sensitive restriction enzyme; (b) terminally dephosphorylating a plurality of the nucleic acids in the sample; (c) contacting the sample from (b) with the first modification-sensitive restriction enzyme under conditions that allow for cleavage of at least some of the first modification-sensitive restriction sites in the nucleic acids in the sample; and (d) contacting the sample from (c) with adapters under conditions that allow for the ligation of the adapters to a 5′ and 3′ end of a plurality of the nucleic acids of interest; thereby generating a sample enriched for nucleic acids of interest that are adapter-ligated on their 5′ and 3′ ends.

The disclose provides methods of enriching a sample for nucleic acids of interest comprising (a) providing a sample comprising nucleic acids of interest and nucleic acids targeted for depletion, wherein at least a subset of the nucleic acids targeted for depletion comprise a plurality of recognition sites for a modification-sensitive restriction enzyme; (b) terminally dephosphorylating a plurality of the nucleic acids in the sample; (c) contacting the sample from (b) with the modification-sensitive restriction enzyme under conditions that allow for the cleavage of the modification-sensitive restriction sites in the nucleic acids in the sample, thereby generating nucleic acids with exposed terminal phosphates; and (d) contacting the sample with an exonuclease under conditions that allow for the successive removal of nucleotides from a phosphorylated end of a nucleic acid; thereby generating a sample enriched for nucleic acids of interest.

The disclose provides methods of enriching a sample for nucleic acids of interest comprising (a) providing a sample comprising nucleic acids of interest and nucleic acids targeted for depletion, wherein at least a subset of the nucleic acids of interest or a subset of the nucleic acids targeted for depletion comprise a plurality of first recognition sites for a first modification-sensitive restriction enzyme, and wherein activity of the first modification-sensitive restriction enzyme is blocked by modification of a nucleotide within or adjacent to its cognate recognition site; (b) terminally dephosphorylating a plurality of the nucleic acids in the sample; (c) contacting the sample from (b) with the first modification-sensitive restriction enzyme under conditions that allow for cleavage of at least some of the first modification-sensitive restriction sites in the nucleic acids in the sample; and (d) contacting the sample from (c) with adapters under conditions that allow for the ligation of the adapters to a 5′ and 3′ end of a plurality of the nucleic acids of interest; thereby generating a sample enriched for nucleic acids of interest that are adapter-ligated on their 5′ and 3′ ends.

The disclose provides methods of enriching a sample for nucleic acids of interest comprising (a) providing a sample comprising nucleic acids of interest and nucleic acids targeted for depletion, wherein at least the nucleic acids targeted for depletion comprise a plurality of recognition sites for a modification-sensitive restriction enzyme; (b) contacting the sample with adapters under conditions that allow for the ligation of the adapters to a 5′ and 3′ end of a plurality of the nucleic acids in the sample; and (c) contacting the sample from (b) with the modification-sensitive restriction enzyme under conditions that allow for the cleavage of the modification-sensitive restriction sites in the nucleic acids in the sample; thereby generating a sample enriched for nucleic acids of interest that are adapter-ligated on their 5′ and 3′ ends.

The disclosure provides methods of depleting nucleic acids targeted for depletion by digestion of the nucleic acids targeted for depletion, thereby enriching a sample for nucleic acids of interest.

The disclosure provides methods of depleting nucleic acids targeted for depletion by digestion of the nucleic acids targeted for by differential adapter attachment to the nucleic acids targeted for depletion and the nucleic acids of interest, thereby enriching a sample for nucleic acids of interest.

The disclosure provides methods of depleting nucleic acids targeted for depletion by without the use of size selection.

The disclosure provides methods of depleting nucleic acids targeted for depletion without the use of modification-sensitive target binding, thereby enriching a sample for nucleic acids of interest. In some embodiments, the methods of depleting nucleic acids targeted for depletion do not use CpG sensitive targeted binding.

In some embodiments, a method of the disclosure comprising a modification-sensitive restriction enzyme is used as a stand-alone method to enrich a sample for nucleic acids of interest. In alternative embodiments, methods of the disclosure that are based on differences in nucleotide modification are combined with one or more additional methods of sample enrichment. In some embodiments, any of the enrichment methods disclosed herein are combined with any other additional enrichment method disclosed herein. In some embodiments, the additional method is a nucleotide modification based method. In some embodiments, the additional method employs libraries of guide nucleic acids (gNAs) and nucleic acid-guided nucleases. In some embodiments, the additional method is a combination of a nucleotide modification based enrichment method and an enrichment method that employs libraries of guide nucleic acids (gNAs) and nucleic acid-guided nucleases. In some embodiments, the additional method depletes the nucleic acids targeted for depletion by digestion of the nucleic acids targeted for depletion. In some embodiments, the additional method depletes the nucleic acids targeted for depletion by differential adapter attachment using the methods of the disclosure. In some embodiments, the additional method depletes the nucleic acids targeted for depletion without the use of size selection. In some embodiments, the additional method depletes the nucleic acids targeted for depletion without the use of modification-sensitive targeted binding. In some embodiments, the additional method depletes the nucleic acids targeted for depletion without the use of CpG sensitive targeted binding.

Unless defined otherwise herein, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

Numeric ranges are inclusive of the numbers defining the range.

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se.

The term “nucleic acid,” as used herein, refers to a molecule comprising one or more nucleic acid subunits. A nucleic acid can include one or more subunits selected from adenosine (A), cytosine (C), guanine (G), thymine (T) and uracil (U), and modified versions of the same. A nucleic acid comprises deoxyribonucleic acid (DNA), ribonucleic acid (RNA), and combinations, or derivatives thereof. A nucleic acid may be single-stranded and/or double-stranded.

The nucleic acids comprise “nucleotides”, which, as used herein, is intended to include those moieties that contain purine and pyrimidine bases, and modified versions of the same.

The term “nucleic acids” and “polynucleotides” are used interchangeably herein. Polynucleotide is used to describe a nucleic acid polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively). DNA and RNA have a deoxyribose and ribose sugar backbones, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

“Modified nucleotides” include, but are not limited to, methylated purines or pyrimidines, acylated purines or pyrimidines, alkylated riboses or other heterocycles. Exemplary modifications include, but are not limited to, cytosine modifications, for example 5-methylcytosine, 5-hydroxymethlcytosine, 5-formylcytosine, 5-carboxylcytosine, glucosythydroxymethylcytosine or 3-methylcytosine.

The term “cleaving,” sometimes also referred to as “cutting”, as used herein, refers to a reaction that breaks the phosphodiester bonds between two adjacent nucleotides in both strands of a double-stranded DNA molecule, thereby resulting in a double-stranded break in the DNA molecule.

The term “nicking” as used herein, refers to a reaction that breaks the phosphodiester bond between two adjacent nucleotides in only one strand of a double-stranded DNA molecule, thereby resulting in a break in one strand of the DNA molecule.

The term “cleavage site”, as used herein, refers to the site at which a double-stranded DNA molecule has been cleaved.

The terms “capture” and “enrichment” are used interchangeably herein, and refer to the process of selectively isolating a nucleic acid region containing: sequences of interest, targeted sites of interest, sequences not of interest, or targeted sites not of interest. In some embodiments, a sample is enriched for sequences of interest, or sequences of interest a captured by selectively depleting sequences that are not of interest. Isolating a nucleic acid region can in some cases be achieved by selectively altering the nucleic acid region of interest in such a way that it is amenable to downstream applications. For example, an isolated nucleic acid can be one which has selectively had adapters ligated to the 5′ and 3′ ends of the nucleic acid.

The term “next-generation sequencing” refers to the so-called parallelized sequencing-by-synthesis or sequencing-by-ligation platforms, for example, those currently employed by Illumina, Life Technologies, and Roche, etc. Next-generation sequencing methods may also include nanopore sequencing methods or electronic-detection based methods such as from Oxford Nanopore, or Ion Torrent technology commercialized by Life Technologies.

### Samples

Nucleic acids isolated or derived from any sort of sample are considered within the scope of the methods of the disclosure.

In some embodiments of the methods of the disclosure, the sample is a biological sample, a clinical sample, a forensic sample or an environmental sample. Clinical and forensic samples include, but are not limited to, whole blood, plasma, serum, tears, saliva, mucous, cerebrospinal fluid, teeth, bone, fingernails, feces, urine tissue and biopsy samples.

In some embodiments, the sample is a metagenomic sample (a sample that contains more than one species of organisms). In some embodiments, a metagenomic sample comprises a sample isolated or derived from organisms that are host to other non-host organisms (e.g., a mammal with one or more viruses, bacteria, fungi or eukaryotic parasites). In some embodiments, a metagenomic sample comprises a sample of microbial communities (e.g., a biofilm).

In some embodiments, the nucleic acids in the sample are fragmented. In some embodiments, the nucleic acids of interest and the nucleic acids targeted for depletion are fragmented.

In some embodiments, the nucleic acids in the sample are about 20 to about 5000 base pairs (bp) in length, about 20 to about 1000 bp in length, about 20 to about 500 bp in length, about 20 to about 400 bp in length, about 20 to about 300 bp in length, about 20 to about 200 bp in length, about 20 to 100 bp in length, about 50 to about 5000 bp in length, about 50 to about 1000 bp in length, about 50 to about 500 bp in length, about 50 to about 400 bp in length, about 50 to about 300 bp in length, about 50 to about 200 bp in length, about 50 to 100 bp in length, about 100 to about 5000 bp in length, about 100 to about 1000 bp in length, about 100 to about 500 bp in length, about 100 to about 400 bp in length, about 100 to about 300 bp in length, about 100 to about 200 bp in length. In some embodiments, the nucleic acids in the sample are about 50 to about 1000 bp in length. In some embodiments, the nucleic acids in the sample are about 50 to about 500 bp in length. In some embodiments, the nucleic acids in the sample are about 100 to about 500 bp in length.

### Nucleic Acids of Interest

Provided herein are methods that can be used to enrich for nucleic acids of interest in a sample for a variety of applications including, but not limited to, amplification, cloning, high-throughput sequencing, detection and quantification of nucleic acids in the sample.

In some embodiments, the nucleic acids of interest comprise at least one recognition site for at least a first modification-sensitive restriction enzyme. In some embodiments, the nucleic acids of interest comprise a plurality of recognition sites for at least a first modification-sensitive restriction enzyme. In some embodiments, the nucleic acids of interest comprise a plurality of recognition sites for each of a first and a second modification-sensitive restriction enzyme. In some embodiments, the activity of the first and/or second modification-sensitive restriction enzyme is blocked by modification of a nucleotide within or adjacent to its cognate restriction site. In some embodiments, the first and/or second modification-sensitive restriction enzyme is active at a recognition site comprising at least one modified nucleotide within or adjacent to the recognition and is not active at a recognition site that does not comprise at least one modified nucleotide within or adjacent to the recognition site. In some embodiments, only the nucleic acids of interest and not the nucleic acids targeted for depletion comprise one or more restriction sites for at least a first modification-sensitive restriction enzyme. In some embodiments, both the nucleic acids of interest and the nucleic acids targeted for depletion comprise a plurality of recognition sites for a first, and optionally a second, modification-sensitive restriction enzyme, but differ in the frequency in which the recognition sites comprise modified nucleotides adjacent to or within the recognition site. In some embodiments, the nucleic acids of interest comprise a plurality of recognition sites for more than two (i.e., at least 3, 4, 5, 6, 7, 8, 9 or 10) modification-sensitive restriction enzymes. In some embodiments, the nucleic acids of interest and the nucleic acids targeted for depletion each comprise a plurality of recognition sites for more than two (i.e., at least 3, 4, 5, 6, 7, 8, 9 or 10) modification-sensitive restriction enzymes.

In some exemplary embodiments, the nucleic acids of interest are from species that lacks CpG methylation or has low levels of CpG methylation (e.g. a non-host species such as a virus, fungus or bacterium). Conversely, in such embodiments the nucleic acids targeted for depletion are from a species which has higher levels of CpG methylation, such as a mammal (e.g. a human). The person of ordinary skill will be able to select a modification sensitive restriction enzyme which has a recognition site containing one or more CG dimers, and whose activity is blocked by the presence of CpG methylation, and use the methods of the disclosure to enrich for nucleic acids of interest.

In some exemplary embodiments, the nucleic acids of interest are from species that lacks CpG methylation or has low levels of CpG methylation (e.g. a non-host species such as a virus, fungus or bacterium). Conversely, in such embodiments the nucleic acids targeted for depletion are from a species which has higher levels of CpG methylation, such as a mammal (e.g. a human). The person of ordinary skill will be able to select a modification sensitive restriction enzyme which has a recognition site containing one or more CG dimers, and whose activity is specific to the presence of CpG methylation within or adjacent to the recognition site, and use the methods of the disclosure to enrich for nucleic acids of interest.

In some embodiments, the nucleic acids of interest are genomic sequences (genomic DNA). In some embodiments, the nucleic acids of interest are mammalian genomic sequences. In some embodiments, the nucleic acids of interest are eukaryotic genomic sequences. In some embodiments, the nucleic acids of interest are prokaryotic genomic sequences. In some embodiments, the sequences of interest are viral genomic sequences. In some embodiments, the nucleic acids of interest are bacterial genomic sequences. In some embodiments, the nucleic acids of interest are plant genomic sequences. In some embodiments, the nucleic acids of interest are microbial genomic sequences. In some embodiments, the sequences of interest are genomic sequences from a parasite, for example a eukaryotic parasite. In some embodiments, the nucleic acids of interest are genomic sequences from a pathogen, for example a bacterium, a virus or a fungus. In some embodiments, the nucleic acids of interest are genomic sequences from a plurality of bacterial, viral or fungal species.

In some embodiments, the nucleic acids of interest can be a genomic fragment, comprising a region of the genome, or the whole genome itself. In one embodiment, the genome is a DNA genome. In another embodiment, the genome is a RNA genome.

In some embodiments, the nucleic acids of interest comprise repetitive sequences. Exemplary but non-limiting repetitive sequences include, but are not limited to mitochondrial sequences, ribosomal sequences, centromeric sequences, Alu elements, long interspersed nuclear elements (LINE) and short interspersed nuclear elements (SINE).

In some embodiments, the nucleic acids of interest are from a eukaryotic or prokaryotic organism; from a mammalian organism or a non-mammalian organism; from an animal or a plant; from a bacteria or virus; from an animal parasite; from a pathogen.

In some embodiments, the nucleic acids of interest are from a species of bacteria. In one embodiment, the bacteria are tuberculosis-causing bacteria.

In some embodiments, the nucleic acids of interest are from a virus.

In some embodiments, the nucleic acids of interest are from a species of fungi.

In some embodiments, the nucleic acids of interest are from a species of algae.

In some embodiments, the nucleic acids of interest are from any mammalian parasite.

In some embodiments, the nucleic acids of interest are obtained from any mammalian parasite. In one embodiment, the parasite is a worm. In another embodiment, the parasite is a malaria-causing parasite. In another embodiment, the parasite is a Leishmaniasis-causing parasite. In another embodiment, the parasite is an amoeba.

In some embodiments, the nucleic acids of interest are from a pathogen.

In some embodiments, the nucleic acids of interest are about 20 to about 5000 bp in length, about 20 to about 1000 bp in length, about 20 to about 500 bp in length, about 20 to about 400 bp in length, about 20 to about 300 bp in length, about 20 to about 200 bp in length, about 20 to about 100 bp in length, about 50 to about 5000 bp in length, about 50 to about 1000 bp in length, about 50 to about 500 bp in length, about 50 to about 400 bp in length, about 50 to about 300 bp in length, about 50 to about 200 bp in length, about 50 to about 100 bp in length, about 100 to about 5000 bp in length, about 100 to about 1000 bp in length, about 100 to about 500 bp in length, about 100 to about 400 bp in length, about 100 to about 300 bp in length, about 100 to about 200 bp in length. In some embodiments, the nucleic acids of interest are about 50 to about 1000 bp in length. In some embodiments, the nucleic acids of interest are about 50 to about 500 bp in length. In some embodiments, the nucleic acids of interest are about 100 to about 500 bp in length.

In some embodiments, the nucleic acids of interest comprise less than 70%, less than 60%, less than 50%, less than 40%, less than 30%, less than 20%, less than 10%, less than 5%, less than 4%, less than 3%, less than 2% or less than 1% of the total nucleic acids in the sample.

In some exemplary embodiments, the nucleic acids of interest comprise less than 50% of the total nucleic acids in the sample.

In some exemplary embodiments, the nucleic acids of interest comprise less than 30% of the total nucleic acids in the sample.

In some exemplary embodiments, the nucleic acids of interest comprise less than 5% of the total nucleic acids in the sample.

In some embodiments, the nucleic acids of interest comprise at least 0.5%, at least 1% at least 2%, at least 3%, at least 4%, at least 5%, at least 6%, at least 7%, at least 8% at least 9%, at least 10%, at least 15%, at least 20%, at least 25%, at least 30%, at least 35%, at least 40%, at least 45% or at least 50% of the total nucleic acids in the sample.

### Nucleic Acids Targeted for Depletion

Provided herein are methods that can be used to deplete nucleic acids from a sample, producing a sample enriched for nucleic acids of interest that can be used for a variety of applications including, but not limited to, amplification, cloning, high-throughput sequencing, detection and quantification of nucleic acids in the sample.

In some embodiments, the nucleic acids targeted for depletion comprise at least one recognition site for at least a first modification-sensitive restriction enzyme. In some embodiments, the nucleic acids targeted for depletion comprise a plurality of recognition sites for at least a first modification-sensitive restriction enzyme. In some embodiments, the nucleic acids targeted for depletion comprise a plurality of recognition sites for each of a first and a second modification-sensitive restriction enzyme. In some embodiments, the activity of the first and/or second modification-sensitive restriction enzyme is blocked by modification of a nucleotide within or adjacent to its cognate restriction site. In some embodiments, the first and/or second modification-sensitive restriction enzyme is active at a recognition site comprising at least one modified nucleotide within or adjacent to the its recognition site and is not active at a recognition site that does not comprise at least one modified nucleotide within or adjacent to the recognition site. In some embodiments, only the nucleic acids targeted for depletion and not the nucleic acids of interest comprise one or more restriction sites for at least a first modification-sensitive restriction enzyme. In some embodiments, both the nucleic acids of interest and the nucleic acids targeted for depletion comprise a plurality of recognition sites for a first, and optionally a second, modification-sensitive restriction enzyme, but differ in the frequency in which the recognition sites comprise modified nucleotides adjacent to or within the recognition site. In some embodiments, the nucleic acids targeted for depletion comprise a plurality of recognition sites for more than two (i.e., at least 3, 4, 5, 6, 7, 8, 9 or 10) modification-sensitive restriction enzymes. In some embodiments, the nucleic acids of interest and the nucleic acids targeted for depletion each comprise a plurality of recognition sites for more than two (i.e., at least 3, 4, 5, 6, 7, 8, 9 or 10) modification-sensitive restriction enzymes.

In some exemplary embodiments, nucleic acids targeted for depletion comprise human RNA or DNA. In some cases, all human nucleic acids are targeted for depletion.

In some exemplary embodiments, the nucleic acids targeted for depletion are from a host species such as a mammal (e.g. a human) that has elevated levels of CpG methylation compared to the nucleic acids of interest. The person of ordinary skill will be able to select a modification sensitive restriction enzyme which has a recognition site containing one or more CG dimers, and whose activity is blocked by the presence of CpG methylation, and use the methods of the disclosure to deplete nucleic acids targeted for depletion resulting in a sample that is enriched for nucleic acids of interest.

In some exemplary embodiments, the nucleic acids targeted for depletion are from a host species such as a mammal (e.g. a human) that has elevated levels of CpG methylation compared to the nucleic acids of interest. The person of ordinary skill will be able to select a modification sensitive restriction enzyme which has a recognition site containing one or more CG dimers, and whose activity is specific to the presence of CpG methylation within or adjacent to the recognition site, and use the methods of the disclosure to deplete nucleic acids targeted for depletion resulting in a sample that is enriched for nucleic acids of interest.

In some embodiments, the nucleic acids targeted for depletion are abundant genomic sequences, such as sequences from the genome or genomes of the most abundant species in a sample. In some embodiments, the most abundant species in the sample is a human.

In some embodiments, the nucleic acids targeted for depletion can be a genomic fragment, comprising a region of the genome, or the whole genome itself. In one embodiment, the genome is a DNA genome. In another embodiment, the genome is a RNA genome.

In some embodiments, the nucleic acids s targeted for depletion are from any mammalian organism. In one embodiment, the mammal is a human. In another embodiment, the mammal is a livestock animal, for example a horse, a sheep, a cow, a pig, or a donkey. In another embodiment, a mammalian organism is a domestic pet, for example a cat, a dog, a gerbil, a mouse, a rat. In another embodiment, the mammal is a type of a monkey.

In some embodiments, the nucleic acids targeted for depletion are from any bird or avian organism. An avian organism includes but is not limited to chicken, turkey, duck and goose.

In some embodiments, the nucleic acids targeted for depletion are from an insect. Insects include, but are not limited to honeybees, solitary bees, ants, flies, wasps or mosquitoes.

In some embodiments, the nucleic acids targeted for depletion are from a plant. In one embodiment, the plant is rice, maize, wheat, rose, grape, coffee, fruit, tomato, potato, or cotton.

In some embodiments, the nucleic acids targeted for depletion comprise repetitive DNA. In some embodiments, the nucleic acids of interest comprise abundant DNA. In some embodiments, the nucleic acids targeted for depletion comprise mitochondrial DNA. In some embodiments, the nucleic acids targeted for depletion comprise ribosomal DNA. In some embodiments, the nucleic acids targeted for depletion comprise centromeric DNA. In some embodiments, the nucleic acids targeted for depletion comprise DNA comprising Alu elements (Alu DNA). In some embodiments, the nucleic acids targeted for depletion comprise long interspersed nuclear elements (LINE DNA). In some embodiments, the nucleic acids targeted for depletion comprise short interspersed nuclear elements (SINE DNA). In some embodiments, the abundant DNA comprises ribosomal DNA.

In some embodiments, the nucleic acids targeted for depletion comprise single nucleotide polymorphisms (SNPs), short tandem repeats (STRs), cancer genes, inserts, deletions, structural variations, exons, genetic mutations, or regulatory regions.

In some embodiments, the nucleic acids targeted for depletion comprise transcriptionally active sequences. For example, transcriptionally active sequences comprises sequences of promoters and of transcriptionally active genes. According to some embodiments, transcriptionally active regions of a genome have higher levels of nucleotide modification than transcriptionally silent regions of a genome. According to some exemplary embodiments, the genome is a mammalian genome, and the nucleotide modification comprises CpG methylation. According to some exemplary embodiments, the genome is a human genome, and the nucleotide modification comprises CpG methylation.

In some embodiments, the nucleic acids targeted for depletion comprise nucleic acids that are common or prevalent in a subject. For example, the depleted nucleic acids can comprise nucleic acids common to all cell types, or more abundant in typical or healthy cells. Following depletion, the remaining nucleic acids to be analyzed can then comprise less common or less prevalent nucleic acids, such as cell type-specific nucleic acids. These less common nucleic acids can be signals of cell death, including cell death of one or more particular cell types. Such signals can be indicative of infections, cancers, and other diseases. In some cases, the signals are signals of cancer-related apoptosis in a particular tissue or tissues. Nucleic acids in a sample isolated or derived from a mixed population of cells can be enriched for nucleic acids from a particular cell type using differences in nucleotide modification between cell types and the methods of the disclosure.

In some embodiments, the nucleic acids targeted for depletion are about 20 to about 5000 bp in length, about 20 to about 1000 bp in length, about 20 to about 500 bp in length, about 20 to about 400 bp in length, about 20 to about 300 bp in length, about 20 to about 200 bp in length, about 20 to about 100 bp in length, about 50 to about 5000 bp in length, about 50 to about 1000 bp in length, about 50 to about 500 bp in length, about 50 to about 400 bp in length, about 50 to about 300 bp in length, about 50 to about 200 bp in length, about 50 to about 100 bp in length, about 100 to about 5000 bp in length, about 100 to about 1000 bp in length, about 100 to about 500 bp in length, about 100 to about 400 bp in length, about 100 to about 300 bp in length, or about 100 to about 200 bp in length. In some embodiments, the nucleic acids targeted for depletion are about 50 to about 1000 bp in length. In some embodiments, the nucleic acids targeted for depletion are about 50 to about 500 bp in length. In some embodiments, the nucleic acids of interest are about 100 to about 500 bp in length.

In some embodiments, the nucleic acids targeted for depletion comprise at least 5%, at least 10%, at least 20%, at least 30%, at least 40%, at least 50%, at least 55%, at least 60% at least 65%, at least 70%, at least 75%, at least 80%, at least 85%, at least 90%, at least 91%, at least 92%, at least 93%, at least 94%, at least 95%, at least 96%, at least 97%, at least 98% or at least 99% of the total nucleic acids in the sample.

### Host/Non-Host Nucleic Acids

In some embodiments, the nucleic acids of interest comprise non-host nucleic acids, and the nucleic acids targeted for depletion comprise host nucleic acids.

In some exemplary embodiments, the host is a vertebrate, and the non-host is a virus, bacterium or fungus. In some embodiments, the vertebrate is a human. In some embodiments, the nucleotide modification comprises CpG, CpC, CpA or CpT methylation, which occurs more frequently in the host genome than the non-host genome. The person of ordinary skill will be able to select a modification sensitive restriction enzyme which has a recognition site containing one or more CG, CC, CA or CT dimers, and whose activity is blocked by the presence of methylation, and use the methods of the disclosure to deplete host nucleic acids targeted for depletion resulting in a sample that is enriched non-host nucleic acids. In some embodiments, the host is a eukaryote. In some embodiments, the host is a mammal, a bird, a reptile or an insect. In some embodiments, the host is a plant. Exemplary mammals include, but are not limited to, a human, a cow, a horse, a sheep, a pig, a monkey, a dog, a cat, a rabbit, a rat, a mouse or a gerbil. In some embodiments, the host is a plant. Exemplary plants include, but are not limited to, agricultural plants such as corn, wheat, rice, tobacco, tomato, orange, apple and almond.

In some embodiments, the host is a human.

In some embodiments, the non-host comprises multiple species of organisms. In some embodiments, the non-host is a single species of organisms. In some embodiments, the non-host comprises a bacterium, a fungus, a virus or a eukaryotic parasite. In some embodiments, the non-host is a pathogen.

### Nucleotide Modifications

Provided herein are methods of enriching a sample for nucleic acids of interest relative to nucleic acids targeted for depletion, comprising using differences in nucleotide modification between the nucleic acids of interest and the nucleic acids targeted for depletion. Any type of nucleotide modification is envisaged as within the scope of the disclosure. Exemplary but non-limiting examples of nucleotide modifications of the disclosure are described below.

Nucleotide modifications used by the methods of the disclosure can occur on any nucleotide (adenine, cytosine, guanine, thymine or uracil, e.g.). These nucleotide modifications can occur on deoxyribonucleic acids (DNA) or ribonucleic acids (RNA). These nucleotide modifications can occur on double or single stranded DNA molecules, or on double or single stranded RNA molecules.

In some embodiments, the nucleotide modification comprises adenine modification or cytosine modification.

In some embodiments, the adenine modification comprises adenine methylation. In some embodiments, the adenine methylation comprises N⁶-methyladenine (6 mA). N⁶-methyladenine (6 mA) is present in both prokaryotic and eukaryotic genomes. The abundance of 6 mA methylation in a genome varies based on species. For example, the abundance of 6 mA is generally lower in mammalian and plant genomes than in prokaryotic genomes. In some cases, the abundance of 6 mA is at least 1,000× higher in a prokaryotic genome when compared to a mammalian or plant genome. In some embodiments, the location of 6 mA methylation in a genome varies based on species. For example, the location of 6 mA methylated nucleotides (within a particular restriction enzyme recognition site, e.g.) depends on the activity of methyltransferases, whose expression and activity varies by species. 6 mA methylation can thus be used to differentiate between eukaryotic and prokaryotic genomes in a sample comprising multiple genomes and selectively enrich for sequences from one genome over the other using the methods of the disclosure.

In some embodiments, the adenine methylation comprises Dam methylation. Dam methylation is a type of DNA nucleotide modification that is carried out by the Deoxyadenosine methylase. Deoxyadenosine methylase (also referred to as DNA adenine methyltransferase, or Dam methylase) is an enzyme that transfers a methyl group from S-adenosylmethionine (SAM) to the N6 position of the adenine residues in the sequence 5′-GATC-3 to generate 6 mA. Dam methylation, and the Dam methylase, are found in prokaryotes and bacteriophages.

In some embodiments, the adenine methylation comprises EcoKI methylation. EcoKI methylation is a type of DNA nucleotide modification that is carried out by the EcoKI methylase. The EcoKI methylase modifies adenine residues in the sequences AAC(N₆)GTGC (SEQ ID NO: 1) and GCAC(N₆)GTT (SEQ ID NO: 2). EcoKI methylase, and EcoKI methylation, are found in prokaryotes.

In some embodiments, the adenine modification comprises adenine modified at N⁶ by glycine (momylation). Momylation changes adenine for N6-(1-acetamido)-adenine. Momylation occurs in viruses, for example bacteriophages.

In some embodiments, the modification comprises cytosine modification. In some embodiments, the abundance and type of cytosine modification in a genome varies based on species. In some embodiments, the location of cytosine modifications (within a particular restriction enzyme recognition site, e.g.) in a genome varies based on species.

In some embodiments, the cytosine modification comprises 5-methylcytosine (5mC), 5-hydroxymethlcytosine (5hmC), 5-formylcytosine (5fC), 5-carboxylcytosine (5caC), 5-glucosylhydroxymethyltosine (5ghmC) or 3-methylcytosine (3mC).

In some embodiments, the cytosine modification comprises cytosine methylation. In some embodiments, the cytosine methylation comprises 5-methylcytosine (5mC) or N4-methylcytosine (4mC).

In some embodiments, 4mC cytosine methylation is found in bacteria. In some embodiments, the bacteria are thermophilic bacteria, for example thermophilic eubacteria or thermophilic archaea.

In some embodiments, the cytosine methylation comprises Dcm methylation. Dcm methylation is a type of methylation that is carried out by the Dcm methylase. In Dcm methylation, the Dcm methylase (encoded by the DNA-cytosine methyltransferase, or dcm gene) methylates the internal (second) cytosine residues in the sequences CCAGG and CCTGG at the C5 position (5mC). Dcm methylase, and Dcm methylation, are found in bacteria such as E. coli.

In some embodiments, the cytosine methylation comprises DNMT1 methylation, DNMT3A methylation or DNMT3B methylation. DNMT1 (DNA methyltransferase 1), DNMT3A (DNA methyltransferase 3 alpha), and DNMT3B (DNA methyltransferase 3 beta) are mammalian methyltransferases that mediate methylation of CpG, CpA, CpT and CpC cytosines.

In some embodiments, the cytosine methylation comprises CpG methylation, CpA methylation, CpT methylation, CpC methylation or a combination thereof. CpG methylation, CpA methylation, CpT methylation, CpC can be found in mammals. While methylated cytosines are frequently found at CpG sites in mammals, non-CpG sites such as CpA, CpT and CpC can also be methylated. In some embodiments, non-CpG methylation is restricted to specific cell types, including, but not limited to, pluripotent stem cells, oocytes and cells of the nervous system. In some embodiments, non-CpG cytosine methylation is mediated by the DNMT3A and DNTM3B methyltransferases. In some embodiments, the cytosine is methylated at the C5 position (5mC). CpA, CpT and CpC methylation can thus be used to distinguish between nucleic acids isolated or derived from different cell types in a sample of mixed cell types.

In some embodiments, the cytosine methylation comprises CpG methylation. CpG methylation in mammals is mediated by the DNMT1, DNMT3A and DNMT3B DNA methyltransferases. DNMT1 primarily binds to hemi-methylated DNA at CpG sites. After DNA replication, the newly synthesized strand lacks methylation, while the parental strain retains a methylated nucleotide. DNMT1 binds to hemi-methylated CpG sites produced by DNA replication and methylates the cytosine on the newly synthesized strand. DNMT3A and DNMT3B do not require hemi-methylated DNA to bind, and show equal affinity for both hemi- and non-methylated CpG sites. In some embodiments, DNMT1, DNMT3A and DNMT3B mediate 5mC methylation. In mammals, CpG methylation occurs more frequently at transcriptionally active sites in the genome, such as in the promoters of active genes. CpG methylation can thus be used to selectively differentiate between active and inactive regions in a mammalian genome. For example, CpG methylation can be used to selectively target an active region in a mammalian genome for depletion using the methods of the disclosure.

In some embodiments, the cytosine modification comprises 5-hydroxymethylcytosine (5hmC). 5hmC is an oxidized derivative of 5mC. 5hmC can be found in viruses (e.g., bacteriophages) as well as some mammalian tissues (for example, brains).

In some embodiments, the cytosine modification comprises 5-formylcytosine (5fC). 5-formylcytosine is an oxidized derivative of 5mC. 5mC is oxidized to 5-hydroxymethylcytosine (5hmC), which is then oxidized to 5fC. In some embodiments, each of these oxidation steps are carried out by Ten-eleven translocation (TET) enzymes. In some embodiments, 5fC is found in mammalian genomes.

In some embodiments, the cytosine modification comprises 5-carboxylcytosine (5caC). 5caC is the final oxidized derivative of 5mC. 5mC is oxidized to 5hmC, which is then oxidized to 5fC, then 5caC, by the TET family of enzymes. In some embodiments, 5caC is found in mammalian genomes.

In some embodiments, the cytosine modification comprises 5-glucosylhydroxymethylcytosine. In some embodiments 5-glucosylhydroxymethylcytosine is found in viruses. In some embodiments, the viruses are bacteriophages. In some embodiments, the viruses are a species of non-host and the viral nucleic acids are nucleic acids of interest in a sample.

In some embodiments, the cytosine modification comprises 3-methylcytosine.

### Modification Sensitive Restriction Enzymes

Provided herein are methods of enriching a sample for nucleic acids of interest relative to nucleic acids targeted for depletion, comprising using differences in nucleotide modification between the nucleic acids of interest and the nucleic acids targeted for depletion that are recognized by one or more modification-sensitive restriction enzymes. Any type of restriction enzyme that is sensitive to any of the nucleotide modifications described herein is within the scope of the disclosure.

In some embodiments of the methods of the disclosure, the methods employ at least a first modification-sensitive restriction enzyme and a second modification-sensitive restriction enzyme. In some embodiments, the first and second modification-sensitive restriction enzymes are the same. In some embodiments, the first and second modification-sensitive restriction enzymes are not the same. In some embodiment, the first or second modification-sensitive restriction enzyme is a single species of restriction enzyme (e.g., Alul, or McrBC, but not both). In some embodiments, the first or second modification-sensitive restriction enzyme is a mixture of 2 or more species of modification- sensitive restriction enzymes (e.g., a mixture of FspEI and AbaSI). In some embodiments of the methods of the disclosure the first or second modification-sensitive restriction enzyme comprises a mixture of at least 2, at least 3, at least 4, at least 5, at least 6, at least 7, at least 8, at least 9 or at least 10 or more species of modification-sensitive restriction enzymes. In some embodiments of the methods of the disclosure, more than two different methods are combined, each using a different modification-sensitive restriction enzyme or cocktail of modification-sensitive restriction enzymes.

The term “modification-sensitive restriction enzyme”, as used herein, refers to a restriction enzyme that is sensitive to the presence of modified nucleotides within or adjacent to the recognition site for the restriction enzyme. The modification-sensitive restriction enzyme can be sensitive to modified nucleotides within the recognition site itself. The modification-sensitive restriction enzyme can be sensitive to modified nucleotides that are adjacent to the recognition site, for example, within 1-50 nucleotides, 5′ or 3′ of the recognition site. The modification-sensitive restriction enzyme can be sensitive to both modified nucleotides within the recognition site and modified nucleotides adjacent to the recognition site. The term “recognition site”, as used herein, refers to a site within a polynucleotide that contains a specific sequence, which is recognized by a restriction enzyme. The restriction enzyme cuts within the recognition site, or nearby to the recognition site, in the polynucleotide. In some embodiments, the restriction enzyme cuts within 1-105 nucleotides of the recognition site. In some embodiments, a restriction enzyme recognizes a pair of recognition half-sites that can be as much as 3 kilobases apart or more in the polynucleotide. In some embodiments, the restriction enzyme recognizes a specific sequence (the recognition site) in the polynucleotide. In some embodiments, the recognition site is between 3-20 bp in length. In some embodiments, the recognition site is palindromic.

Nucleotide modifications of the disclosure can be within the recognition site itself, or comprise nucleotides adjacent to the recognition site (for example, within 1-50 nucleotides, 5′ or 3′ of the recognition site, or both).

In some embodiments, the modification-sensitive restriction enzymes is sensitive to a single modified nucleotide within or adjacent to the recognition site.

In some embodiments, the modification-sensitive restriction enzymes is sensitive to multiple modified nucleotides within or adjacent to the recognition site.

In some embodiments, the modification-sensitive restriction enzymes is sensitive to a particular type or types of modification (e.g., methylation, hydroxymethylation or carboxylation) on one or more nucleotides within or adjacent to the recognition site.

In some embodiments, the modification-sensitive restriction enzyme is sensitive to modification at a particular nucleotide or nucleotides within or adjacent to the recognition site.

In some embodiments, the modification-sensitive restriction enzyme is sensitive to a particular spatial arrangement of modified nucleotides within or adjacent to the recognition site. For example, a modification-sensitive restriction enzyme can be sensitive to a pair of modifications, on opposite strands, and one or two nucleotides apart, within the recognition site in a DNA polynucleotide.

In some embodiments, the modification-sensitive restriction enzyme is blocked by the presence of one or more modified nucleotides within or adjacent to the recognition site. Modification-sensitive restriction enzymes that are blocked by the presence of modified nucleotides cut at recognition sites that do not contain modified nucleotides, and do not cut or cut at reduced levels at recognition sites that contain modified nucleotides.

Modification-sensitive restriction enzymes whose activity is blocked by modified nucleotides include enzymes whose activity is blocked or reduced by any sort of modified nucleotide, or any combination of modified nucleotides, within or adjacent to the recognition site. Exemplary modifications capable of blocking or reducing the activity of modification-sensitive restriction enzymes include, but are not limited to, N⁶-methyladenine, 5-methylcytosine (5mC), 5-hydroxymethlcytosine (5hmC), 5-formylcytosine (5fC), 5-carboxylcytosine (5caC), 5-glucosylhydroxymethycytosine, 3-methylcytosine (3mC), N4-methylcytosine (4mC) or combinations thereof. Exemplary modifications capable of blocking modification-sensitive restriction enzymes include modifications mediated by Dam, Dcm, EcoKI, DNMT1, DNMT3A, DNMT3B and TET enzymes.

In some embodiments, the modification comprises Dam methylation. Restriction enzymes that are blocked by Dam methylation include, but are not limited to, the enzymes in table 1 below:

TABLE 1
Restriction enzymes whose
activity is blocked by Dam methylation

| Restriction Enzyme | Recognition Site |
|---|---|
| AlwI | GGATC |
| BcgI | CGATCNNNNTGC (SEQ ID NO: 3) |
| BclI | TGATCA |
| BsaBI | GATCNNNATC (SEQ ID NO: 4) |
| BspDI | ATCGATC |
| BspEI | TCCGGATC |
| BspHI | TCATGATC |
| ClaI | ATCGATC |
| DpnII | GATC |
| HphI | GGTGATC |
| Hpy 188I | TCNGATC |
| Hpy 188III | TCNNGATC |
| MboI | GATC |
| MboII | GAAGATC |
| NruI | TCGCGATC |
| Nt.AlwI | GGATCNNNNN (SEQ ID NO: 5) |
| Taqα I | TCGATC |
| XbaI | TCTAGATC |

In some embodiments, the modification comprises Dcm methylation. Restriction enzymes that are blocked by Dcm methylation include, but are not limited to, the enzymes in table 2 below:

TABLE 2
Restriction enzymes whose
activity is blocked by Dcm methylation

| Restriction Enzyme | Recognition Site |
|---|---|
| Acc65I | GGTACCWGG |
| AlwNI | CAGNNCCTGG (SEQ ID NO: 6) |
| ApaI | GGGCCCWGG |
| AvaI | CYCGRG |
| AvaII | GGWCCWGG |
| BanI | GGYRCCWGG |
| BsaI | GAGACCWGG |
| BsaHI | GRCGCCWGG and GRCGYC |
| BslI | CCWGGNNNNGG (SEQ ID NO: 7) |
| BsmFI | GGGACT |
| BssKI | CCWGG |
| BstXI | CCAGGNNNNTGG (SEQ ID NO: 8) |
| EaeI | YGGCCAGG |
| Esp3I | CGTCTC |
| EcoO109I | RGGNCCTGG |
| MscI | TGGCCAGG |
| NlaIV | GGNNCCWGG |
| PflMI | CCAGGNNNTGG (SEQ ID NO: 9) |
| PspGI | CCWGG |
| PspOMI | GGGCCCWGG |
| Sau96I | GGNCCWGG |
| ScrFI | CCWGG |
| SexAI | ACCWGGT |
| SfiI | GGCCWGGNNGGCC (SEQ ID NO: |
| | 10) or GGCCNNNNNGGCCWGG (SEQ |
| | ID NO: 11) |
| SfoI | GGCGCC |
| StuI | AGGCCTGG |

In some embodiments, the modification comprises CpG methylation. Restriction enzymes that are blocked by CpG methylation include, but are not limited to, the enzymes in table 3 below:

TABLE 3
Restriction enzymes whose
activity is blocked by CpG methylation

| Restriction Enzyme | Recognition Site |
|---|---|
| Aat II | GACGTC |
| AccII | CGCG |
| AciI | CCGC |
| AcII | AACGTT |
| AfeI | AGCGCT |
| AgeI | ACCGGT |
| Aor13HI | TCCGGA |
| Aor51HI | AGCGCT |
| AscI | GGCGCGCC |
| AsiSI | GGCGCGCC |
| AluI | AGCT |
| AvaI | CYCGRG |
| BceAI | ACGGC |
| BmgBI | CACGTC |
| BsaI | GAGACCWGG |
| BsaHI | GRCGCCWGG and GRCGYC |
| BsiEI | CGRYCG |
| BsiWI | CGTACG |
| BsmBI | CGTCTC |
| BspDI | ATCGAT |
| BspT104I | TTCGAA |
| BsrFalphaI | RCCGGY |
| BssHII | GCGCGC |
| BstBI | TTCGAA |
| BstUI | CGCG |
| Cfr10I | RCCGGY |
| ClaI | ATCGAT |
| CpoI | CGGWCCG |
| EagI | CGGCCG |
| Esp3I | CGTCTC |
| Eco52I | CGGCCG |
| FauI | CCCGC |
| FseI | GGCCGGCC |
| FspI | TGCGCA |
| HaeII | RGCGCY |
| HgaI | GACGC |
| HhaI | GCGC |
| HpaII | CCGG |
| HpyCH4IV | ACGT |
| Hpy99I | CGWCG |
| KasI | GGCGCC |
| MluI | ACGCGT |
| NaeI | GCCGGC |
| NgoMIV | GCCGGC |
| NotI | GCGGCCGC |
| NruI | TCGCGA |
| Nt.BsmAI | GTCTC |
| Nt.CviPII | CCD |
| NsbI | TGCGCA |
| PmaCI | CACGTG |
| Psp1406I | AACGTT |
| PluTI | GGCGCC |
| PmlI | CACGTG |
| PvuI | CGATCG |
| RsrII | CGGWCCG |
| SacII | CCGCGG |
| SalI | GTCGAC |
| SmaI | CCGGG |
| SnaBI | TACGTA |
| SfoI | GGCGCC |
| SgrAI | CRCCGGYG |
| SmaI | CCCGGGG |
| SrfI | GCCCGGGC |
| Sau3AI | GATC |
| TspMI | CCCGGG |
| ZraI | GACGTC |

In some embodiments, a modification-sensitive restriction enzyme is active at a recognition site comprising at least one modified nucleotide and is not active at a recognition site that does not comprise at least one modified nucleotide. For example, a modification-sensitive restriction enzyme will cleave at a recognition site containing one or modified nucleotides, but will not cleave a recognition site that does not contain one or more modified nucleotides.

Exemplary modifications recognized by modification-sensitive restriction enzymes that cleave at recognition sites comprising one or more modified nucleotides include, but are not limited to, N⁶-methyladenine, 5-methylcytosine (5mC), 5-hydroxymethlcytosine (5hmC), 5-formylcytosine (5fC), 5-carboxylcytosine (5caC), 5-glucosylhydroxymethylcytosine, 3-methylcytosine (3mC), N4-methylcytosine (4mC) or combinations thereof. Exemplary modifications recognized modification-sensitive restriction enzymes that specifically cleave recognition sites comprising one or more modified nucleotides include modifications mediated by Dam, Dcm, EcoKI, DNMT1, DNMT3A, DNMT3B and TET enzymes.

Exemplary but non-limiting modification-sensitive restriction enzymes that cleave at a recognition site comprising one or more modified nucleotides within or adjacent to the recognition site are listed in Table 4 below.

TABLE 4
Restriction enzymes that cleave
recognition sites comprising modified nucleotides

| Restriction | | |
|---|---|---|
| Enzyme | Recognition Site | Modification |
| AbaSI | 5′-^{ghm}CN₁₁₋₁₃/N₉₋₁₀ G-3′ | ^{ghm}C = 5-glucosylhydroxymethylcytosine; |
| | (SEQ ID NOs: 12-15) | *C = 5-glucosylhydroxymethylcytosine, |
| | 3′-GN₉₋₁₀/N₁₁₋₁₃*C-5′ | 5-hydroxymethylcytosine, |
| | (SEQ ID NOs: 16-19) | 5-methylcytosine or cytosine |
| DpnI | G^{m}ATC | adenine methylation |
| FspEI | 5′-C^{m}CN₁₂-3′ | ^{m}C = 5-methylcytosine or |
| | (SEQ ID NO: 20) | 5-hydroxymethylcytosine |
| | 3′-G GN₁₆-5′ | |
| | (SEQ ID NO: 21) | |
| LpnPI | 5′-C^{m}CDGN₁₀-3′ | ^{m}C = 5-methylcytosine or |
| | (SEQ ID NO: 22) | 5-hydroxymethylcytosine |
| | 3′-G GHCN₁₄-5′ | |
| | (SEQ ID NO: 23) | |
| MspJI | 5′-^{m}CNNRN₉-3′ | ^{m}C = 5-methylcytosine or |
| | (SEQ ID NO: 24) | 5-hydroxymethylcytosine |
| | 3′-GNNYN₁₃-5′ | |
| | (SEQ ID NO: 25) | |
| McrBC | (G/A)^{m}C half site, | ^{m}C = 5-methylcytosine, |
| | separated by up to | 5-hydroxymethylcytosine, |
| | 3 kb, optimal | N4-methylcytosine, on one or both |
| | separation 55-103 bp | strands |

In some embodiments, the modification comprises 5-glucosylhydroxymethylcytosine and the modification-sensitive restriction enzyme comprises AbaSI. AbaSI cleaves an AbaSI recognition site comprising a glucosylhydroxymethylcytosine, and does not cleave an AbaSI recognition site that does not comprise a glucosylhydroxymethylcytosine.

In some embodiments, the nucleotide modification comprises 5-hydroxymethylcytosine and the modification-sensitive restriction enzyme comprises AbaSI and T4 phage 0-glucosyltransferase. T4 Phage 0-glucosyltransferase specifically transfers the glucose moiety of uridine diphosphoglucose (UDP-Glc) to the 5-hydroxymethylcytosine (5-hmC) residues in double-stranded DNA, for example, within the AbaSI recognition site, making a glucosylhydroxymethylcytosine modified AbaSI recognition site. AbaSI cleaves an AbaSI recognition site comprising glucosylhydroxymethylcytosine and does not cleave an AbaSI recognition site that does not comprise a glucosylhydroxymethylcytosine.

In some embodiments, the nucleotide modification comprises methylcytosine and the modification-sensitive restriction enzyme comprises McrBC. McrBC cleaves McrBC sites comprising methylcytosines, and does not cleave McrBC sites that do not comprise methylcytosines. The McrBC site can be modified with methylcytosines on one or both DNA strands. In some embodiments, McrBC also cleaves McrBC sites comprising hydroxymethylcytosines on one or both DNA strands. In some embodiments, the McrBC half sites are separated by up to 3,000 nucleotides. In some embodiments, the McrBC half sites are separated by 55-103 nucleotides.

In some embodiments, the modification comprises adenine methylation and the methods comprise digestion with DpnI. DpnI cleaves a GATC recognition site when the adenines on both strands of the GATC recognition are methylated. In some embodiments, DpnI GATC recognition sites comprising both adenine methylation and cytosine modification occur in bacterial DNA, but not in mammalian DNA. These recognition sites comprising both methylated adenines and modified cytosines can be selectively cleaved by DpnI in a sample (e.g., of mixed bacterial and mammalian DNA), and then treated with T4 polymerase to replace methylated adenines and modified cytosines at the cleaved ends with unmodified adenines and cytosines. T4 polymerase catalyzes the synthesis of DNA in the 5′ to 3′ direction, in the presence of a template, primer and nucleotides. T4 polymerase will incorporate unmodified nucleotides into the newly synthesized DNA. This produces a sample that now comprises unmodified cytosines in the nucleic acids of interest and modified cytosines in the nucleic acids targeted for depletion. These differences in modified cytosines can be used to enrich for nucleic acids of interest using the methods of the disclosure.

### Phosphatases

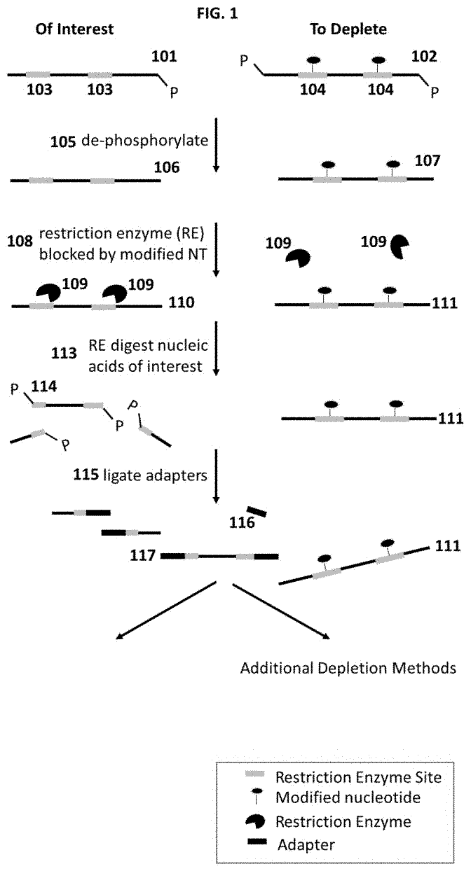

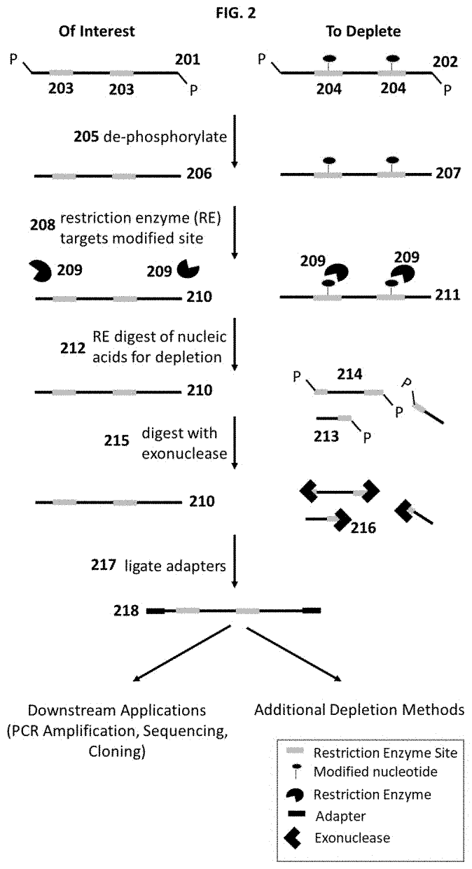

In some embodiments of the methods of the disclosure, the nucleic acids in the sample are terminally dephosphorylated, so that contacting the nucleic acids in the sample with a modification-sensitive restriction enzyme produces either nucleic acids of interest or nucleic acids targeted for depletion with exposed terminal phosphates than can be used in the methods of the disclosure to enrich the sample for nucleic acids of interest. For example, these exposed terminal phosphates can be used to target the nucleic acids for depletion for degradation by an exonuclease (FIG. 2) or the nucleic acids of interest for adapter ligation (FIG. 1).

As used herein, the term “terminally dephosphorylated” refers to nucleic acids that have had the terminal phosphate groups removed from the 5′ and 3′ ends of the nucleic acid molecule.

In some embodiments, the nucleic acids in the sample are terminally dephosphorylated using a phosphatase. Phosphatases are enzymes that non-specifically catalyze the dephosphorylation of the 5′ and 3′ ends of DNA and RNA molecules. In some embodiments, the phosphatase is an alkaline phosphatase.

Exemplary phosphatases of the disclosure include, but are not limited to shrimp alkaline phosphatase (SAP), recombinant shrimp alkaline phosphatase (rSAP), calf intestine alkaline phosphatase (CIP) and Antarctic phosphatase.

### Exonucleases

As used herein, the term “exonuclease” refers to a class of enzymes successively remove nucleotides from the 3′ or 5′ ends of a nucleic acid molecule. The nucleic acid molecule can be DNA or RNA. The DNA or RNA can be single stranded or double stranded. Exemplary exonucleases include, but are not limited to Lambda nuclease, Exonuclease I, Exonuclease III and BAL-31. Exonucleases can be used to selectively degrade nucleic acids targeted for depletion using the methods of the disclosure (FIG. 2, e.g.).

In some embodiments, Exonuclease III is used to degrade cleaved DNA targeted for depletion while leaving uncut DNA of interest intact. Exonuclease III can initiate unidirectional 3′>5′ degradation of one DNA strand by using blunt end or 5′ overhangs that have terminal phosphates, yielding single-stranded DNA and nucleotides; it is not active on single-stranded DNA or DNA lacking terminal phosphates, and thus 3′ overhangs, such Y shaped adapter ends, are resistant to degradation. As a result, intact double-stranded DNA fragments of interest that are uncut by modification-sensitive restriction enzymes and lack terminal phosphates are not digested by Exonuclease III, while DNA molecules targeted for depletion that have been cleaved by modification-sensitive restriction enzymes are degraded by Exonuclease III.

In some embodiments, Exonuclease I is used to degrade cleaved DNA targeted for depletion while leaving uncut DNA of interest intact. In some embodiments, a sample of nucleic acid fragments (e.g. single stranded DNA) is dephosphorylated and cut with a modification-sensitive restriction enzyme that cuts the nucleic acids targeted for depletion but does not cut the nucleic acids of interest. Exonuclease I degrades single-stranded DNA in a 3′ to 5′ direction.

In some embodiments, Lambda nuclease (Lambda Exonuclease) is used to degrade cleaved DNA targeted for depletion while leaving uncut DNA of interest intact. In some embodiments, a sample of nucleic acid fragments (e.g. DNA) is dephosphorylated and cut with a modification-sensitive restriction enzyme that cuts the nucleic acids targeted for depletion but does not cut the nucleic acids of interest. Lambda nuclease is a highly processive 5′ to 3′ exonuclease. Its preferred substrate is 5′ phosphorylated double stranded DNA, and it degrades non-phosphorylated DNA at greatly reduced rates. Thus, intact, dephosphorylated nucleic acids of interest are protected from lambda nuclease, while cut nucleic acids targeted for depletion that have exposed 5′ phosphates are degraded.

In some embodiments, Exonuclease BAL-31 is used degrade cleaved DNA targeted for depletion while leaving the uncut DNA of interest intact. In some embodiments, a sample of nucleic acid fragments (e.g. DNA) is dephosphorylated and cut with a modification-sensitive restriction enzyme that cuts the nucleic acids targeted for depletion but does not cut the nucleic acids of interest. The sample is contacted with a modification-sensitive restriction enzyme, which cuts the nucleic acids targeted for depletion and leaves the nucleic acids of interest intact. The resulting products are contacted with Exonuclease BAL-31. Exonuclease BAL-31 has two activities: double-stranded DNA exonuclease activity, and single-stranded DNA/RNA endonuclease activity. The double-stranded DNA exonuclease activity allows BAL-31 to degrade DNA from open ends on both strands, thus reducing the size of double-stranded DNA. The longer the incubation, the greater the reduction in size of the double-stranded DNA, making it useful for depleting medium to large DNA fragments (>200 bp). In some embodiments, the 3′ ends of the nucleic acids are tailed with poly-dG using terminal transferase. It was noted that the single-stranded endonuclease activity of BAL-31 allows it to digest poly-A, -C or -T very rapidly, but is extremely low in digesting poly-G. Because of this nature, adding single-stranded poly-dG at 3′ ends of the libraries serves as a protection from being degraded by BAL-31. As a result, DNA molecules that have been poly-dG tailed and cleaved by a modification-sensitive restriction enzyme can be degraded by BAL-31; while intact DNA libraries are not digested by BAL-31 due to their 3′ end poly-dG protection and/or lack of terminal phosphates.

In some embodiments of the methods of the disclosure, the methods comprise contacting the sample with an exonuclease under conditions that allow for the successive removal of nucleotides from a phosphorylated end of a nucleic acid. In some embodiments, the nucleic acids in the sample are terminally dephosphorylated. In some embodiments, contacting the sample with the exonuclease comprises contacting the sample with the exonuclease following cleavage of the nucleic acids in the sample with a modification-sensitive restriction enzyme that exposes terminal phosphates on the ends of the cleaved nucleic acids in the sample. In some embodiments, the nucleic acids in the sample with the exposed terminal phosphates comprise nucleic acids targeted for depletion. In some embodiments, the exonuclease depletes at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the nucleic acids targeted for depletion from the sample.

### Adapters

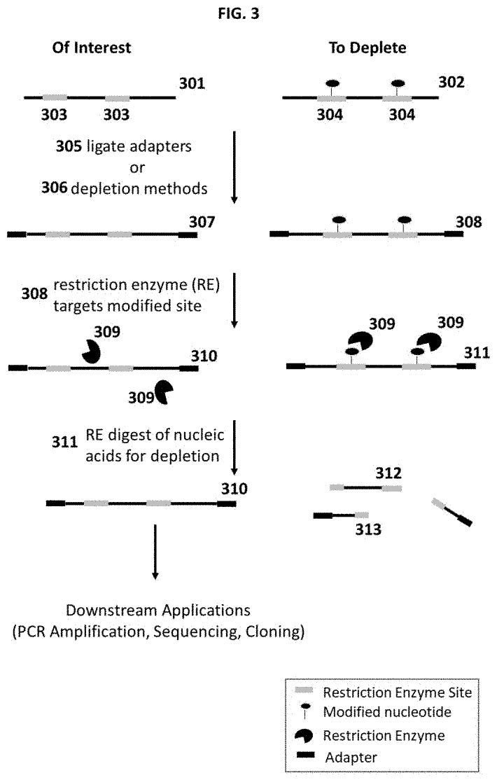

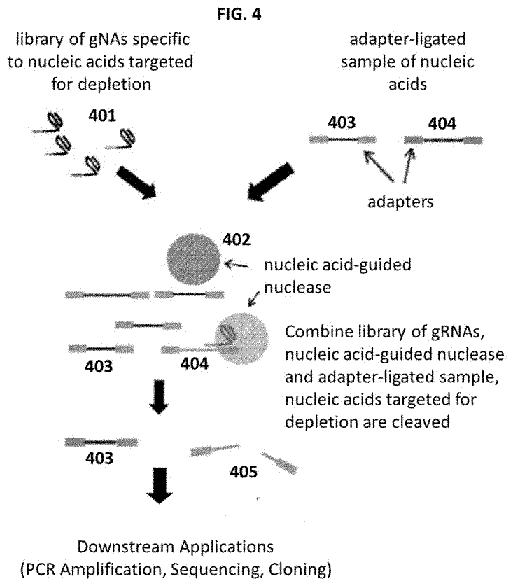

The disclosure provides adapters that are ligated to the 5′ and 3′ ends of the nucleic acids in the sample or the nucleic acids of interest. In some embodiments of the methods of the disclosure, adapters are ligated to all the nucleic acids in the sample, and then differences in nucleotide modification are used to selectively cleave the nucleic acids targeted for depletion, producing nucleic acids of interest that are adapter ligated on both ends and nucleic acids targeted for depletion that are adapter ligated on one end (FIG. 3, FIG. 4). In some embodiments, differences in nucleotide modification are used to selectively deplete the nucleic acids targeted for depletion, and then adapters are ligated to the nucleic acids of interest (FIG. 2). In some embodiments, differences in nucleotide modification are used to produce nucleic acids of interest with exposed terminal phosphates, which are used to ligate adapters to the nucleic acids of interest (FIG. 1).

In some embodiments of the methods of the disclosure, adapters are ligated to the 5′ and 3′ ends of the nucleic acids in the sample. In some embodiments, the adapters further comprise intervening sequence between the 5′ terminal end and/or the 3′ terminal end. For example an adapter can further comprise a barcode sequence.

In some embodiments the adapter is a nucleic acid that is ligatable to both strands of a double-stranded DNA molecule.

In some embodiments, adapters are ligated prior to depletion/enrichment. In other embodiments, adapters are ligated at a later step.

In some embodiments the adapters are linear. In some embodiments the adapters are linear Y-shaped. In some embodiments the adapters are linear circular. In some embodiments the adapters are hairpin adapters. In some embodiments, the adapters comprise a polyG sequence.

In various embodiments the adapter may be a hairpin adapter i.e., one molecule that base pairs with itself to form a structure that has a double-stranded stem and a loop, where the 3′ and 5′ ends of the molecule ligate to the 5′ and 3′ ends of the double-stranded DNA molecule of the fragment, respectively.

Alternately, the adapter may be a Y-adapter ligated to one end or to both ends of a fragment, also called a universal adapter. Alternately, the adapter may itself be composed of two distinct oligonucleotide molecules that are base paired with one another. Additionally a ligatable end of the adapter may be designed to be compatible with overhangs made by cleavage by a restriction enzyme, or it may have blunt ends or a 5′ T overhang. In some embodiments, the restriction enzyme is a modification-sensitive restriction enzyme.

The adapter may include double-stranded as well as single-stranded molecules. Thus the adapter can be DNA or RNA, or a mixture of the two. Adapters containing RNA may be cleavable by RNase treatment or by alkaline hydrolysis.

Adapters can be 10 to 100 bp in length although adapters outside of this range are usable without deviating from the present disclosure. In specific embodiments, the adapter is at least 10 bp, at least 15 bp, at least 20 bp, at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 85 bp, at least 90 bp, or at least 95 bp in length.

In some embodiments, the adapter-ligated nucleic acids of interest and nucleic acids targeted for depletion range from about 20 to about 5000 bp in length, about 20 to about 1000 bp in length, about 20 to about 500 bp in length, about 20 to about 400 bp in length, about 20 to about 300 bp in length, about 20 to about 200 bp in length, about 20 to 100 bp in length, about 50 to about 5000 bp in length, about 50 to about 1000 bp in length, about 50 to about 500 bp in length, about 50 to about 400 bp in length, about 50 to about 300 bp in length, about 50 to about 200 bp in length, about 50 to 100 bp in length, about 100 to about 5000 bp in length, about 100 to about 1000 bp in length, about 100 to about 500 bp in length, about 100 to about 400 bp in length, about 100 to about 300 bp in length, about 100 to about 200 bp in length. In some embodiments, the adapter-ligated nucleic acids of interest and nucleic acids targeted for depletion range from about 50 to about1000 bp in length. In some embodiments, the adapter-ligated nucleic acids of interest and nucleic acids targeted for depletion range from about 50 to about 500 bp in length. In some embodiments, the adapter-ligated nucleic acids of interest and nucleic acids targeted for depletion range from about 100 to about 500 bp in length. In some embodiments, the adapter-ligated nucleic acids of interest and nucleic acids targeted for depletion range from 50-300 bp in length.

In some embodiments, an adapter may comprise an oligonucleotide designed to match a nucleotide sequence of a particular region of the host genome, e.g., a chromosomal region whose sequence is deposited at NCBI's Genbank database or other databases. Such an oligonucleotide may be employed in an assay that uses a sample containing a test genome, where the test genome contains a binding site for the oligonucleotide. In further examples the fragmented nucleic acid sequences may be derived from one or more DNA sequencing libraries. An adapter may be configured for a next generation sequencing platform, for example for use on an Illumina sequencing platform or for use on an IonTorrents platform, or for use with Nanopore technology.

In some embodiments, the adapters comprise sequencing adapters (e.g., Illumina sequencing adapters). In some embodiments, the adapters comprise unique molecular identifier (UMI) sequences. In some embodiments, the UMI sequences comprise a sequence that is unique to each original nucleic acid molecule (e.g., a random sequence). This can allow quantification of nucleic amounts, free from sequencing bias. In some embodiments, the adapters comprise “barcode” sequences. In some embodiments, the barcode sequences comprise a barcode sequence that is shared among nucleic acid molecules from a particular source (such as a subject, patient, environmental sample, partition (e.g., droplet, well, bead)). This can allow pooling of sequencing information for subsequent analysis, and can allow detection and elimination of cross-contamination. In some embodiments, the adapters comprise multiple distinct sequences, such as a UMI unique to each nucleic acid molecule, a barcode shared among nucleic acid molecules from a particular source, and a sequencing adapter.

### Depletion

The nucleic acids targeted for depletion can be depleted by a variety of approaches.

The nucleic acids targeted for depletion can be depleted by differential adapter attachment. In some embodiments, adapters are attached to nucleic acids of a sample, and subsequently one or more adapters are removed from nucleic acids targeted for depletion based on their modification status. For example, nucleic acids targeted for depletion with adapters attached to both ends can be cleaved by a modification-sensitive restriction enzyme, thereby producing nucleic acids targeted for depletion with adapters attached to only one end. Subsequent steps (e.g., amplification) can be used to target only nucleic acids with adapters attached to both ends, thereby depleting the nucleic acids targeted for depletion. In another example, the nucleic acids of the sample are treated (e.g., by dephosphorylation) such that only cleaved nucleic acids are able to have adapters attached; subsequently, nucleic acids of interest can be cleaved by a modification-sensitive restriction enzyme (e.g., thereby exposing a phosphate group) and adapters can be attached. Subsequent steps (e.g., amplification) can be used to target only nucleic acids with adapters attached, thereby depleting the nucleic acids targeted for depletion.

The nucleic acids targeted for depletion can be depleted by digestion. For example, the nucleic acids of the sample are treated (e.g., by dephosphorylation) such that only cleaved nucleic acids are able to be digested (e.g., by an exonuclease). Nucleic acids targeted for depletion can be cleaved by a modification-sensitive restriction enzyme, thereby rendering them able to be digested. Subsequent digestion, such as with an exonuclease, can then be used to deplete the nucleic acids targeted for depletion.

The nucleic acids targeted for depletion can be depleted by size selection. For example, a modification-sensitive restriction enzyme can be used to cleave either the nucleic acids of interest or the nucleic acids targeted for depletion, and subsequently the nucleic acids of interest can be separated from the nucleic acids targeted for depletion based on size differences due to the cleavage.

In some cases, the nucleic acids targeted for depletion are depleted without the use of size selection.

The nucleic acids targeted for depletion can be depleted by targeted binding. For example, a modification-sensitive binding domain (e.g., a methylation-sensitive antibody or DNA binding domain) can be used to bind to and separate either the nucleic acids targeted for depletion or the nucleic acids of interest based on their modification status. As used herein, a “modification-sensitive binding domain” refers to a protein, protein fragment or fusion protein which binds to nucleic acids in a modification-sensitive fashion, but, unlike the modification-sensitive restriction enzymes disclose herein, does not cut the nucleic acids. “Modification-sensitive targeted binding” refers to the binding of nucleic acids by a modification-sensitive binding domain. In some exemplary embodiments, the binding of the modification-sensitive binding domain to the nucleic acids is sufficiently stable to allow for the selective binding of either the nucleic acids targeted for depletion or the nucleic acids of interest followed by subsequent purification, for example by co-immunoprecipitation, or conjugation of the modification-sensitive binding domain to beads or a column.

In some cases, the nucleic acids targeted for depletion are depleted without the use of modification-sensitive targeted binding. In some cases, the nucleic acids targeted for depletion are depleted without the use of CpG sensitive targeted binding.

### Methods

Protocol 1: Exemplary methods of the application described herein are depicted in FIG. 1. A sample of nucleic acids comprising nucleic acids of interest (101) and nucleic acids targeted for depletion (102) is terminally dephosphorylated (105) to produce unphosphorylated nucleic acids of interest (106) and nucleic acids targeted for depletion (107). In some embodiments, the nucleic acids are fragmented prior to dephosphorylation. In some embodiments, the nucleic acids in the sample are terminally dephosphorylated with a phosphatase, for example recombinant shrimp alkaline phosphatase (rSAP). In some embodiments, both the nucleic acids of interest and the nucleic acids targeted for depletion comprise one or more recognition sites for a modification-sensitive restriction enzyme (103, 104, respectively). In the nucleic acids of interest, the recognition sites for the modification-sensitive restriction enzyme do not comprise modified nucleotides (103), or alternatively, contain modified nucleotides less frequently than the corresponding recognition sites of the nucleic acids targeted for depletion. In the nucleic acids targeted for depletion, the recognition sites for the modification-sensitive restriction enzyme comprise modified nucleotides within or adjacent to the restriction site (104), or alternatively, comprise modified nucleotides more frequently than the corresponding recognition sites of the nucleic acids of interest. Activity of the modification-sensitive restriction enzyme (109) is blocked by the presence of modified nucleotides within or adjacent to its cognate recognition site (108), thereby targeting the activity of the modification-sensitive restriction enzyme to the nucleic acids of interest (compare 110 and 111). In some embodiments, the modification-sensitive restriction enzyme (109) comprises AatII, AccII, Aor13HI, Aor51HI, BspT104I, BssHII, Cfr10I, ClaI, CpoI, Eco52I, HaeII, HapII, HhaI, MluI, NaeI, NotI, NruI, NsbI, PmaCI, Psp1406I, PvuI, SacII, SalI, SmaI, SnaBI, AluI or Sau3AI. In some embodiments, the modification-sensitive restriction enzyme (109) comprises AluI or Sau3AI. Digesting the sample with the modification-sensitive restriction enzyme (113) produces nucleic acids of interest with terminal phosphates at the 5′ and 3′ ends of the terminal phosphates (114). These terminal phosphates are used to ligate adapters (115, ligation step; 116, adapters) to the ends of the nucleic acids of interest, producing nucleic acids of interest that are adapter ligated on both ends (117). In contrast, the nucleic acids targeted for depletion are not adapter ligated (111). These adapters can be used for downstream applications, for example adapter-mediated PCR amplification, sequencing (e.g. high throughput sequencing), quantification of the nucleic acids of interest in the sample and/or cloning. This depletes the nucleic acids targeted for depletion by selectively ligating adapters to the nucleic acids of interest. This depletion can be accomplished without the use of size selection. Alternatively the adapter ligated nucleic acids of interest are subjected to one or more of the additional enrichment methods described herein. For example, the adapter ligated nucleic acids are subjected to additional modification-dependent enrichment methods of the disclosure (for example, the methods depicted in FIG. 3). Alternatively, or in addition, the adapter ligated nucleic acids are subjected to nucleic acid-guided nuclease based enrichment methods of the disclosure (for example, the methods depicted in FIG. 4).

Protocol 2: Exemplary methods of the application described herein are depicted in FIG. 2. A sample of nucleic acids comprising nucleic acids of interest (201) and nucleic acids targeted for depletion (202) is terminally dephosphorylated (205) to produce unphosphorylated nucleic acids of interest (206) and nucleic acids targeted for depletion (207). In some embodiments, the nucleic acids are fragmented prior to dephosphorylation. In some embodiments, the nucleic acids in the sample are terminally dephosphorylated with a phosphatase, for example recombinant shrimp alkaline phosphatase (rSAP). In some embodiments, both the nucleic acids of interest and the nucleic acids targeted for depletion comprise one or more recognition sites for a modification-sensitive restriction enzyme (203 and 204, respectively). In the nucleic acids of interest, the recognition sites for the modification-sensitive restriction enzyme do not comprise modified nucleotides (203), or alternatively, contain modified nucleotides less frequently than the corresponding recognition sites of the nucleic acids targeted for depletion. In the nucleic acids targeted for depletion, the recognition sites for the modification-sensitive restriction enzyme comprise modified nucleotides within or adjacent to the restriction site (204), or alternatively, comprise modified nucleotides more frequently than the corresponding recognition sites of the nucleic acids of interest. The modification-sensitive restriction enzyme (209) cuts its cognate recognition site when there are one or more modified nucleotides within or adjacent to the recognition site (208), and does not cut its cognate recognition site when the recognition site does not comprise one or more modified nucleotides (208), thereby targeting the activity of the modification-sensitive restriction enzyme to the nucleic acids targeted for depletion (compare 210 and 211). In some embodiments, the modification-sensitive restriction enzyme comprises AbaSI, FspEI, LpnPI, MspJI or McrBC. In some embodiments, the modification-sensitive restriction enzyme is FspEI. In some embodiments, the modification-sensitive restriction enzyme is MspJI. Digestion of the sample with the modification-sensitive restriction enzyme (212) produces nucleic acids targeted for depletion with terminal phosphates one end (213) or both the 5′ and 3′ ends of the nucleic acid (214). In contrast, the nucleic acids of interest, which were not cut by the modification-sensitive restriction enzyme, do not have exposed terminal phosphates at the 5′ and or 3′ ends of the nucleic acids (compare 210 with 213-214). The sample is then digested with an exonuclease (215, digestion step; 216 exonuclease) which uses the terminal phosphates in the nucleic acids targeted for depletion to remove successive nucleotides from the ends of the nucleic acids molecules, thus depleting the nucleic acids targeted for depletion from the sample. This depletion can be accomplished without the use of size selection. Following exonuclease digestion, adapters are ligated to the nucleic acids of interest (217), which, lacking terminal phosphates, have not been digested by the exonuclease. This produces nucleic acids of interest that are adapter ligated on both ends (218). These adapters can be used for downstream applications, for example adapter-mediated PCR amplification, sequencing (e.g. high throughput sequencing), quantification of the nucleic acids of interest in the sample and/or cloning. Alternatively the adapter ligated nucleic acids of interest are subjected to one or more of the additional enrichment methods described herein. For example, the adapter ligated nucleic acids are subjected to additional modification-dependent enrichment methods of the disclosure (for example, the methods depicted in FIG. 3). Alternatively, or in addition, the adapter ligated nucleic acids are subjected to nucleic acid-guided nuclease based enrichment methods of the disclosure (for example, the methods depicted in FIG. 4).

Protocol 3: Exemplary methods of the application described herein are depicted in FIG. 3. A sample of nucleic acids comprising nucleic acids of interest (301) and nucleic acids targeted for depletion (302) is adapter-ligated (305), or is subjected to enrichment methods of the disclosure (306) (e.g., the methods depicted in FIG. 1 or FIG. 2) that produce adapter-ligated nucleic acids of interest (307) and adapter-ligated nucleic acids targeted for depletion (308). In some embodiments, both the nucleic acids of interest and the nucleic acids targeted for depletion comprise one or more recognition sites for a modification-sensitive restriction enzyme (303 and 304, respectively). In the nucleic acids of interest, the recognition sites for the modification-sensitive restriction enzyme do not comprise modified nucleotides (303), or alternatively, contain modified nucleotides less frequently than the corresponding recognition sites of the nucleic acids targeted for depletion. In the nucleic acids targeted for depletion, the recognition sites for the modification-sensitive restriction enzyme comprise modified nucleotides within or adjacent to the restriction site (304), or alternatively, comprise modified nucleotides more frequently than the corresponding recognition sites of the nucleic acids of interest. The modification-sensitive restriction enzyme (309) cuts its cognate recognition site when there are one or more modified nucleotides within or adjacent to the recognition site (308), and does not cut its cognate recognition site when the recognition site does not comprise one or more modified nucleotides (308), thereby targeting the activity of the modification-sensitive restriction enzyme to the nucleic acids targeted for depletion (compare 310 and 311). In some embodiments, the modification-sensitive restriction enzyme comprises AbaSI, FspEI, LpnPI, MspJI or McrBC. In some embodiments, the modification-sensitive restriction enzyme is FspEI. In some embodiments, the modification-sensitive restriction enzyme is MspJI. The sample is digested with the modification-sensitive restriction enzyme (311), producing nucleic acids targeted for depletion that are not adapter ligated (312), or are adapter ligated on only one end (313). This depletes the nucleic acids targeted for depletion by selectively removing adapters from the nucleic acids targeted for depletion. This depletion can be accomplished without the use of size selection. In contrast, the nucleic acids of interest, which were not cut by the modification-sensitive restriction enzyme, are adapter ligated on both ends (contrast 310 with 312-313). These adapters can be used for downstream applications, for example adapter-mediated PCR amplification, sequencing (e.g. high throughput sequencing), quantification of the nucleic acids of interest in the sample and/or cloning.

Protocol 4: Exemplary methods of the application described herein are depicted in FIG. 4. A plurality of gNAs (401) are used to target a nucleic acid-guided nuclease (402) to nucleic acids targeted for depletion (403) in a sample of adapter-ligated nucleic acids. The adapter ligated nucleic acids are generated by any of the methods of enrichment described herein that use modification-sensitive restriction enzymes to deplete nucleic acids targeted for depletion from a sample, either before or after an initial adapter ligation. In this method, the gNAs are specifically targeted to the nuclei acids targeted for depletion (403), and not the nucleic acids of interest (404), which are therefore not cut by the nucleic acid-guided nuclease (402). Cleavage by the nucleic acid-guided nuclease results in nucleic acids targeted for depletion that are adapter ligated on one end (405), and nucleic acids of interest that are adapter ligated on both ends (403). These adapters can be used for downstream applications, for example adapter-mediated PCR amplification, sequencing (e.g. high throughput sequencing), quantification of the nucleic acids of interest in the sample and cloning.

Protocol 5: In some embodiments, the nucleic acid-guided nuclease is a nucleic acid-guided Nickase. A plurality of gNAs are used to target a nucleic acid-guided nickase to nucleic acids targeted for depletion in a sample of adapter-ligated nucleic acids. The adapter ligated nucleic acids are generated by any of the methods of enrichment described herein that use modification-sensitive restriction enzymes to deplete nucleic acids targeted for depletion from a sample, either before or after an initial adapter ligation. In some embodiments, the plurality of gNAs is designed so that all the nucleic acids targeted for depletion will have two gNA binding sites in close proximity (for example, less than 15 bases apart) on opposite DNA strands of a double stranded DNA targeted for depletion. In this embodiment, the nucleic acid-guided Nickase can recognize its target sites on the DNA to be removed and cuts only one strand. For DNA to be depleted, two separate nucleic acid-guided Nickases can cut both strands of the DNA to be depleted in close proximity; only the DNA to be depleted will have two nucleic acid-guided nickase sites in close proximity which creates a double stranded break. If a nucleic acid-guided Nickase, e.g. a CRISPR/Cas system protein Nickase recognizes non-specifically or at low affinity a site on the DNA of interest, it can only cut one strand which would not prevent subsequent PCR amplification or downstream processing of the DNA molecule. In this embodiment, the chances of two gNAs recognizing two sites non-specifically in close enough proximity is negligible (<1×10⁻¹⁴). This embodiment would be particularly useful if regular, CRISPR/Cas system protein -mediated cleavage cuts too much of the DNA of interest.

Protocol 6: In some embodiments, the nucleic acid-guided nuclease is catalytically dead, and the method involves partitioning the nucleic acids targeted for depletion and the nucleic acids of interest in the sample. A plurality of gNAs are used to target a catalytically dead nucleic acid-guided nuclease (e.g., dCas9 or dCpf1 ) to either the nucleic acids targeted for depletion or the nucleic acids of interest in a sample of adapter-ligated nucleic acids. The adapter ligated nucleic acids are generated by any of the methods of enrichment described herein that use modification-sensitive restriction enzymes to deplete nucleic acids targeted for depletion from a sample, either before or after an initial adapter ligation. The catalytically dead nucleic acid-guided nuclease is capable of binding to nucleic acids, but not nicking or cutting the nucleic acids. In some embodiments, the catalytically dead nucleic acid-guided nuclease comprises a tag, such as a biotin tag, which can be used to isolated the catalytically dead nucleic acid-guided nuclease and any molecules to which it is bound. In these embodiments, a plurality of gNAs is developed that hybridize either to the nucleic acids of interest or the nucleic acids targeted for depletion, but not both. This plurality of gNAs and the catalyically dead nucleic-acid guided nuclease are contacted with the sample allowing the catalytically dead nucleic acid-nuclease to bind to either the nucleic acids of interest or the nucleic acids targeted for depletion, depending on the design of the gNAs. Instead of cutting the targeted sequences, this method is used to partition the fragmented nucleic acid sample into two fractions which can each be processed separately. Accordingly, the catalytically dead nucleic-acid guided nuclease partitions the mixture into unbound fragments (e.g., the nucleic acids of interest) and bound fragments (e.g. the nucleic acids targeted for depletion, to which the gNAs are targeted). The bound portion of the target nucleic acid sample is removed by binding of an affinity tag (e.g., biotin) previously attached to the catalytically dead nucleic acid-guided nuclease protein. The bound nucleic acid sequences can be eluted from the protein/gNA complex by denaturing conditions and then amplified and sequenced. Similarly, the unbound nucleic acid sequences can be amplified and sequenced.

Any of the methods described herein can be used as a stand-alone method to deplete nucleic acids targeted for depletion from a sample, thereby enriching for nucleic acids of interest.

Alternatively, the methods described herein can be combined to achieve a greater degree of enrichment than any individual method in alone. In some embodiments, a sample is first enriched using Procotol 1, followed by Protocol 2. In some embodiments, a sample is first enriched using Procotol 1, followed by Protocol 3. In some embodiments, a sample is first enriched using Procotol 1, followed by Protocol 2 and 3. In some embodiments, a sample is first enriched using Procotol 1, followed by any one of Protocols 4-6. In some embodiments, a sample is first enriched using Procotol 1, followed by Protocol 2 and/or 3 and any one of Protocols 4-6.

While particular combinations of methods, and orders of combinations of methods, are described herein, these are in no way intended to limit the ways in which the methods of the disclosure can be combined. Any method of enriching a sample for nucleic acids of interest of the disclosure that produces adapter ligated nucleic acids of interest as a product of the method can be combined with any additional methods of the disclosure that use adapter ligated nucleic acids as its starting substrate.

### Nucleic Acid-Guided Nuclease Based Enrichment Methods

In some embodiments of the methods of the disclosure, the modification-based enrichment methods of the disclosure are combined with nucleic acid-guided nuclease based enrichment methods. Nucleic acid-guided nuclease based enrichment methods are methods that employ nucleic acid-guided nucleases to enrich a sample for sequences of interest. Nucleic acid-guided nuclease based enrichment methods are described in WO/2016/100955, WO/2017/031360, WO/2017/100343, WO/2017/147345 and WO/2018/227025 the contents of each of which are herein incorporated by reference in their entirety.

In some embodiments, the modification-based enrichment methods and the nucleic acid-guided nuclease based enrichment methods of the disclosure deplete different nucleic acids in the sample, thereby achieving a greater degree of enrichment for the nucleic acids of interest than either approach alone. For example, a sample comprises nucleic acids targeted for depletion from a mammalian host genome and nucleic acids of interest from one or more non-host genomes (e.g., bacteria, viruses or parasites). Using the methods of the disclosure to enrich nucleic acids of interest in this sample, modification-based enrichment methods are selected that take advantage of differences in CpG methylation between host and non-host nucleic acids to deplete nucleic acids comprising actively transcribed regions of the mammalian host genome, while nucleic acid-guided nuclease based enrichment methods effectively target regions of repetitive sequence in the mammalian host genome using library of guide nucleic acids (gNAs) that target those regions.

The term “nucleic acid-guided nuclease-gNA complex” refers to a complex comprising a nucleic acid-guided nuclease protein and a guide nucleic acid (gNA, for example a gRNA or a gDNA). For example, the “Cas9-gRNA complex” refers to a complex comprising a Cas9 protein and a guide RNA (gRNA). The nucleic acid-guided nuclease may be any type of nucleic acid-guided nuclease, including but not limited to a wild type nucleic acid-guided nuclease, a catalytically dead nucleic acid-guided nuclease, or a nucleic acid-guided nuclease-nickase.

Pluralities of gNAs

Provided herein are pluralities (interchangeably referred to as libraries, or collections) of guide nucleic acids (gNAs).

The term “guide nucleic acid” refers to a guide nucleic acid (gNA) that is capable of forming a complex with a nucleic acid guided nuclease, and optionally, additional nucleic acid(s). The gNA may exist as an isolated nucleic acid, or as part of a nucleic acid-guided nuclease-gNA complex, for example a Cas9-gRNA complex.

As used herein, a plurality of gNAs denotes a mixture of gNAs containing at least 10² unique gNAs. In some embodiments a plurality of gNAs contains at least 10² unique gNAs, at least 10³ unique gNAs, at least 10⁴ unique gNAs, at least 10⁵ unique gNAs, at least 10⁶ unique gNAs, at least 10⁷ unique gNAs, at least 10⁸ unique gNAs, at least 10⁹ unique gNAs or at least 10¹⁰ unique gNAs. In some embodiments a collection of gNAs contains a total of at least 10² unique gNAs, at least 10³ unique gNAs, at least 10⁴ unique gNAs or at least 10⁵ unique gNAs.

In some embodiments, a collection of gNAs comprises a first NA segment comprising a targeting sequence; and a second NA segment comprising a nucleic acid-guided nuclease system (e.g., CRISPR/Cas system) protein-binding sequence. In some embodiments, the first and second segments are in 5′- to 3′-order'. In some embodiments, the first and second segments are in 3′- to 5′-order'.

In some embodiments, the size of the first segment varies from 12-250 bp, or 12-100 bp, or 12-75 bp, or 12-50 bp, or 12-30 bp, or 12-25 bp, or 12-22 bp, or 12-20 bp, or 12-18 bp, or 12-16 bp, or 14-250 bp, or 14-100 bp, or 14-75 bp, or 14-50 bp, or 14-30 bp, or 14-25 bp, or 14-22 bp, or 14-20 bp, or 14-18 bp, or 14-17 bp, or 14-16 bp, or 15-250 bp, or 15-100 bp, or 15-75 bp, or 15-50 bp, or 15-30 bp, or 15-25 bp, or 15-22 bp, or 15-20 bp, or 15-18 bp, or 15-17 bp, or 15-16 bp, or 16-250 bp, or 16-100 bp, or 16-75 bp, or 16-50 bp, or 16-30 bp, or 16-25 bp, or 16-22 bp, or 16-20 bp, or 16-18 bp, or 16-17 bp, or 17-250 bp, or 17-100 bp, or 17-75 bp, or 17-50 bp, or 17-30 bp, or 17-25 bp, or 17-22 bp, or 17-20 bp, or 17-18 bp, or 18-250 bp, or 18-100 bp, or 18-75 bp, or 18-50 bp, or 18-30 bp, or 18-25 bp, or 18-22 bp, or 18-20 bp, or 19-250 bp, or 19-100 bp, or 19-75 bp, or 19-50 bp, or 19-30 bp, or 19-25 bp, or 19-22 bp across the plurality of gNAs. In some embodiments, the size of the first segment varies from or 15-250 bp, or 30-100 bp, or 20-30bp, or 22-30 bp, or 15-50bp, or 15-75 bp, or 15-100 bp, or 15-125 bp, or 15-150 bp, or 15-175 bp, or 15-200 bp, or 15-225 bp, or 15-250 bp, or 22-50 bp, or 22-75 bp, or 22-100 bp, or 22-125 bp, or 22-150 bp, or 22-175 bp, or 22-200 bp, or 22-225 bp, or 22-250 bp across the plurality of gNAs.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the first segments in the plurality are 15-50 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the first segments in the collection are 15-20 bp.

In some particular embodiments, the size of the first segment is 15 bp. In some particular embodiments, the size of the first segment is 16 bp. In some particular embodiments, the size of the first segment is 17 bp. In some particular embodiments, the size of the first segment is 18 bp. In some particular embodiments, the size of the first segment is 19 bp. In some particular embodiments, the size of the first segment is 20 bp.

In some embodiments, the gNAs and/or the targeting sequence of the gNAs in the plurality of gRNAs comprise unique 5′ ends. In some embodiments, the plurality of gNAs exhibits variability in sequence of the 5′ end of the targeting sequence, across the members of the plurality. In some embodiments, the plurality of gNAs exhibits at least 5%, or at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75% variability in the sequence of the 5′ end of the targeting sequence, across the members of the plurality.

In some embodiments, the 3′ end of the gNA targeting sequence can be any purine or pyrimidine (and/or modified versions of the same). In some embodiments, the 3′ end of the gNA targeting sequence is an adenine. In some embodiments, the 3′ end of the gNA targeting sequence is a guanine. In some embodiments, the 3′ end of the gNA targeting sequence is a cytosine. In some embodiments, the 3′ end of the gNA targeting sequence is a uracil. In some embodiments, the 3′ end of the gNA targeting sequence is a thymine. In some embodiments, the 3′ end of the gNA targeting sequence is not cytosine.

In some embodiments, the plurality of gNAs comprises targeting sequences which can base-pair with a target sequence in the nucleic acids targeted for depletion, wherein the target sequence in the nucleic acids targeted for depletion is spaced at least every 1 bp, at least every 2 bp, at least every 3 bp, at least every 4 bp, at least every 5 bp, at least every 6 bp, at least every 7 bp, at least every 8 bp, at least every 9 bp, at least every 10 bp, at least every 11 bp, at least every 12 bp, at least every 13 bp, at least every 14 bp, at least every 15 bp, at least every 16 bp, at least every 17 bp, at least every 18 bp, at least every 19 bp, 20 bp, at least every 25 bp, at least every 30 bp, at least every 40 bp, at least every 50 bp, at least every 100 bp, at least every 200 bp, at least every 300 bp, at least every 400 bp, at least every 500 bp, at least every 600 bp, at least every 700 bp, at least every 800 bp, at least every 900 bp, at least every 1000 bp, at least every 2500 bp, at least every 5000 bp, at least every 10,000 bp, at least every 15,000 bp, at least every 20,000 bp, at least every 25,000 bp, at least every 50,000 bp, at least every 100,000 bp, at least every 250,000 bp, at least every 500,000 bp, at least every 750,000bp, or even at least every 1,000,000 bp across a genome or transcriptome targeted for depletion in the sample.

In some embodiments, the plurality of gNAs comprises a first NA segment comprising a targeting sequence; and a second NA segment comprising a nucleic acid-guided nuclease system (e.g., CRISPR/Cas system) protein-binding sequence, wherein the gNAs in the plurality can have a variety of second NA segments with various specificities for protein members of the nucleic acid-guided nuclease system (e.g., CRISPR/Cas system). For example a collection of gNAs as provided herein, can comprise members whose second segment comprises a nucleic acid-guided nuclease system (e.g., CRISPR/Cas system) protein-binding sequence specific for a first nucleic acid-guided nuclease system (e.g., CRISPR/Cas system) protein; and also comprises members whose second segment comprises a nucleic acid-guided nuclease system (e.g., CRISPR/Cas system) protein-binding sequence specific for a second nucleic acid-guided nuclease system (e.g., CRISPR/Cas system) protein, wherein the first and second nucleic acid-guided nuclease system (e.g., CRISPR/Cas system) proteins are not the same. In some embodiments a collection of gNAs as provided herein comprises members that exhibit specificity to at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or even at least 20 nucleic acid-guided nuclease system (e.g., CRISPR/Cas system) proteins. In one specific embodiment, a plurality of gNAs as provided herein comprises members that exhibit specificity for a Cas9 protein and another protein selected from the group consisting of Cpf1, Cas3, Cas8a-c, Cas10, CasX, CasY, Cas13, Cas14, Cse1, Csy1, Csn2, Cas4, Csm2, and Cm5. In some embodiments, the nucleic acid-guided nuclease system protein-binding sequences specific for the first and second nucleic acid-guided nuclease system proteins are both 5′ of the first NA segment comprising a targeting sequence. In some embodiments, the nucleic acid-guided nuclease system protein-binding sequences specific for the first and second nucleic acid-guided nuclease system proteins are both 3′ of the first NA segment comprising a targeting sequence. In some embodiments, the nucleic acid-guided nuclease system protein-binding sequence specific for the first nucleic acid-guided nuclease system (e.g., CRISPR/Cas system) protein is 5′ of the first NA segment comprising a targeting sequence and the second nucleic acid-guided nuclease system protein-binding sequences specific for the second nucleic acid-guided nuclease system protein is 3′ of the first NA segment comprising a targeting sequence. The order of the first NA segment comprising a targeting sequence and the second NA segment comprising a nucleic acid-guided nuclease system protein-binding sequence will depend on the nucleic acid-guided nuclease system protein. The appropriate 5′ to 3′ arrangement of the first and second NA segments and choice of nucleic acid-guided nuclease system proteins will be apparent to one of ordinary skill in the art.

In some embodiments the gNAs comprise DNA and RNA. In some embodiments, the gNAs consist of DNA (gDNAs). In some embodiments, the gNAs consist of RNA (gRNAs).

In some embodiments, the gNA comprises a gRNA and the gRNA comprises two sub-segments, which encode for a crRNA and a tracrRNA. In some embodiment, the crRNA does not comprise the targeting sequences plus the extra sequence which can hybridize with tracrRNA. In some embodiments, the crRNA comprises an extra sequence which can hybridize with tracrRNA. In some embodiments, the two sub-segments are independently transcribed. In some embodiments, the two sub-segments are transcribed as a single unit. In some embodiments, the DNA encoding the crRNA comprises the targeting sequence 5′ of the sequence GTTTTAGAGCTATGCTGTTTTG (SEQ ID NO: 26). In some embodiments, the DNA encoding the tracrRNA comprises the sequence

(SEQ ID NO: 27)
GGAACCATTCAAAACAGCATAGCAAGTTAAAATAAGGCTAGTCCGTTATC
AACTTGAAAAAGTGGCACCGAGTCGGTGCTTTTTTT.

### Targeting Sequences

As used herein, a targeting sequence is one that directs the gNA to a target sequence in a nucleic acid targeted for depletion in a sample. For example, a targeting sequence targets a particular sequence, for example the targeting sequence targets a repetitive sequence in a genome targeted for depletion in the sample.

Provided herein are gNAs and pluralities of gNAs that comprise a segment that comprises a targeting sequence.

In some embodiments, the targeting sequence comprises or consists of DNA.

In some embodiments, the targeting sequence comprises or consists of RNA.

In some embodiments, the targeting sequence comprises RNA, and shares at least 70% sequence identity, at least 75% sequence identity, at least 80% sequence identity, at least 85% sequence identity, at least 90% sequence identity, at least 95% sequence identity, or shares 100% sequence identity to a sequence 5′ to a PAM sequence on a sequence of interest, except that the RNA comprises uracils instead of thymines. In some embodiments, the targeting sequence comprises RNA, and shares at least 70% sequence identity, at least 75% sequence identity, at least 80% sequence identity, at least 85% sequence identity, at least 90% sequence identity, at least 95% sequence identity, or shares 100% sequence identity to a sequence 3′ to a PAM sequence on a sequence of interest, except that the RNA comprises uracils instead of thymines. In some embodiments, the PAM sequence is AGG, CGG, TGG, GGG or NAG. In some embodiments, the PAM sequence is TTN, TCN or TGN.

In some embodiments, the targeting sequence comprises DNA, and shares at least 70% sequence identity, at least 75% sequence identity, at least 80% sequence identity, at least 85% sequence identity, at least 90% sequence identity, at least 95% sequence identity, or shares 100% sequence identity to a sequence 5′ to a PAM sequence on a sequence of interest. In some embodiments, the targeting sequence comprises DNA, and shares at least 70% sequence identity, at least 75% sequence identity, at least 80% sequence identity, at least 85% sequence identity, at least 90% sequence identity, at least 95% sequence identity, or shares 100% sequence identity to a sequence 3′ to a PAM sequence on a sequence of interest.

In some embodiments, the targeting sequence comprises RNA and is complementary to the strand opposite to a sequence of nucleotides 5′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 5′ to a PAM sequence. In some embodiments, the targeting sequence comprises RNA and is complementary to the strand opposite to a sequence of nucleotides 3′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 3′ to a PAM sequence. In some embodiments, the PAM sequence is AGG, CGG, TGG, GGG or NAG. In some embodiments, the PAM sequence is TTN, TCN or TGN.

In some embodiments, the targeting sequence comprises DNA and is complementary to the strand opposite to a sequence of nucleotides 5′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 5′ to a PAM sequence. In some embodiments, the targeting sequence comprises DNA and is complementary to the strand opposite to a sequence of nucleotides 3′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 3′ to a PAM sequence. In some embodiments, the PAM sequence is AGG, CGG, TGG, GGG or NAG. In some embodiments, the PAM sequence is TTN, TCN or TGN.

Different CRISPR/Cas system proteins recognize different PAM sequences. PAM sequences can be located 5′ or 3′ of a targeting sequence. For example, Cas9 can recognize an NGG PAM located on the immediate 3′ end of a targeting sequence. Cpf1 can recognize a TTN PAM located on the immediate 5′ end of a targeting sequence. All PAM sequences recognized by all CRISPR/Cas system proteins are envisaged as being within the scope of the disclosure. It will be readily apparent to one of ordinary skill in the art which PAM sequences are compatible with a particular CRISPR/Cas system protein.

### Nucleic Acid-Guided Nucleases

Provided herein are gNAs and pluralities of gNAs comprising a segment that comprises a nucleic acid-guided nuclease protein-binding sequence. The nucleic acid-guided nuclease can be a nucleic acid-guided nuclease system protein (e.g., CRISPR/Cas system). A nucleic acid-guided nuclease system can be an RNA-guided nuclease system. A nucleic acid-guided nuclease system can be a DNA-guided nuclease system.

Methods of the present disclosure can utilize nucleic acid-guided nucleases. As used herein, a “nucleic acid-guided nuclease” is any nuclease that cleaves DNA, RNA or DNA/RNA hybrids, and which uses one or more guide nucleic acids (gNAs) to confer specificity. Nucleic acid-guided nucleases include CRISPR/Cas system proteins as well as non-CRISPR/Cas system proteins.

The nucleic acid-guided nucleases provided herein can be DNA guided DNA nucleases; DNA guided RNA nucleases; RNA guided DNA nucleases; or RNA guided RNA nucleases. The nucleases can be endonucleases. The nucleases can be exonucleases. In one embodiment, the nucleic acid-guided nuclease is a nucleic acid-guided-DNA endonuclease. In one embodiment, the nucleic acid-guided nuclease is a nucleic acid-guided-RNA endonuclease.

A nucleic acid-guided nuclease protein-binding sequence is a nucleic acid sequence that binds any protein member of a nucleic acid-guided nuclease system. For example, a CRISPR/Cas protein-binding sequence is a nucleic acid sequence that binds any protein member of a CRISPR/Cas system.

In some embodiments, the nucleic acid-guided nuclease is selected from the group consisting of CAS Class I Type I, CAS Class I Type III, CAS Class I Type IV, CAS Class II Type II, and CAS Class II Type V. In some embodiments, CRISPR/Cas system proteins include proteins from CRISPR Type I systems, CRISPR Type II systems, and CRISPR Type III systems. In some embodiments, the nucleic acid-guided nuclease is selected from the group consisting of Cas9, Cpf1, Cas3, Cas8a-c, Cas10, Cas13, Cas14, Cse1, Csy 1, Csn2, Cas4, Csm2, Cm5, Csf1, C2c2, CasX, CasY, Cas14 and NgAgo.

In some embodiments, nucleic acid-guided nuclease system proteins (e.g., CRISPR/Cas system proteins) can be from any bacterial or archaeal species.

In some embodiments, the nucleic acid-guided nuclease system proteins (e.g., CRISPR/Cas system proteins) are from, or are derived from nucleic acid-guided nuclease system proteins (e.g., CRISPR/Cas system proteins) from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis Corynebacter diphtheria, Acidaminococcus, Lachnospiraceae bacterium or Prevotella.

In some embodiments, examples of nucleic acid-guided nuclease system (e.g., CRISPR/Cas system) proteins can be naturally occurring or engineered versions.

In some embodiments, naturally occurring nucleic acid-guided nuclease system (e.g., CRISPR/Cas system) proteins include Cas9, Cpf1, Cas3, Cas8a-c, Cas10, CasX, CasY, Cas13, Cas14, Cse1, Csy1, Csn2, Cas4, Csm2, and Cm5. Engineered versions of such proteins can also be employed.

In some embodiments, engineered examples of nucleic acid-guided nuclease (e.g., CRISPR/Cas) system proteins also include nucleic acid-guided nickases (e.g., Cas nickases). A nucleic acid-guided nickase refers to a modified version of a nucleic acid-guided nuclease system protein, containing a single inactive catalytic domain. In one embodiment, the nucleic acid-guided nickase is a Cas nickase, such as Cas9 nickase. A Cas9 nickase may contain a single inactive catalytic domain, for example, either the RuvC- or the HNH-domain. With only one active nuclease domain, the Cas9 nickase cuts only one strand of the target DNA, creating a single-strand break or “nick”. Depending on which mutant is used, the guide NA-hybridized strand or the non-hybridized strand may be cleaved. Nucleic acid-guided nickases bound to 2 gNAs that target opposite strands will create a double-strand break in a target double-stranded DNA. This “dual nickase” strategy can increase the specificity of cutting because it requires that both nucleic acid-guided nuclease/gNA (e.g., Cas9/gRNA) complexes be specifically bound at a site before a double-strand break is formed. Naturally occurring nickase nucleic acid-guided nuclease system proteins can also be employed.

In some embodiments, engineered examples of nucleic acid-guided nuclease system proteins also include nucleic acid-guided nuclease system fusion proteins. For example, a nucleic acid-guided nuclease (e.g., CRISPR/Cas) system protein may be fused to another protein, for example an activator, a repressor, a nuclease, a fluorescent molecule, a radioactive tag, or a transposase.

In some embodiments, the nucleic acid-guided nuclease system protein-binding sequence comprises a gNA (e.g., gRNA) stem-loop sequence.

Different CRISPR/Cas system proteins are compatible with different nucleic acid-guided nuclease system protein-binding sequences. It will be readily apparent to one of ordinary skill in the art which CRISPR/Cas system proteins are compatible with which nucleic acid-guided nuclease system protein-binding sequences.

In some embodiments, a double-stranded DNA sequence encoding the gNA (e.g., gRNA) stem-loop sequence comprises the following DNA sequence on one strand (5′>3′, GTTTTAGAGCTAGAAATAGCAAGTTAAAATAAGGCTAGTCCGTTATCAACTTGAA AAAGTGGCACCGAGTCGGTGCTTTTTTT (SEQ ID NO: 28)), and its reverse-complementary DNA on the other strand (5′>3′, AAAAAAAGCACCGACTCGGTGCCACTTTTTCAAGTTGATAACGGACTAGCCTTAT TTTAACTTGCTATTTCTAGCTCTAAAAC (SEQ ID NO: 29)).

In some embodiments, a single-stranded DNA sequence encoding the gNA (e.g., gRNA) stem-loop sequence comprises the following DNA sequence: (5′>3′, AAAAAAAGCACCGACTCGGTGCCACTTTTTCAAGTTGATAACGGACTAGCCTTAT TTTAACTTGCTATTTCTAGCTCTAAAAC (SEQ ID NO: 29)), wherein the single-stranded DNA serves as a transcription template.

In some embodiments, the gNA (e.g., gRNA) stem-loop sequence comprises the following RNA sequence: (5′>3′, GUUUUAGAGCUAGAAAUAGCAAGUUAAAAUAAGGCUAGUCCGUUAUCAACUU GAAAAAGUGGCACCGAGUCGGUGCUUUUUUU (SEQ ID NO: 30)).

In some embodiments, a double-stranded DNA sequence encoding the gNA (e.g., gRNA) stem-loop sequence comprises the following DNA sequence on one strand (5′>3′, GTTTTAGAGCTATGCTGGAAACAGCATAGCAAGTTAAAATAAGGCTAGTCCGTTA TCAACTTGAAAAAGTGGCACCGAGTCGGTGCTTTTTTTC (SEQ ID NO: 31)), and its reverse-complementary DNA on the other strand (5′>3′, GAAAAAAAGCACCGACTCGGTGCCACTTTTTCAAGTTGATAACGGACTAGCCTTA TTTTAACTTGCTATGCTGTTTCCAGCATAGCTCTAAAAC (SEQ ID NO: 32)).

In some embodiments, a single-stranded DNA sequence encoding the gNA (e.g., gRNA) stem-loop sequence comprises the following DNA sequence: (5′>3′, GAAAAAAAGCACCGACTCGGTGCCACTTTTTCAAGTTGATAACGGACTAGCCTTA TTTTAACTTGCTATGCTGTTTCCAGCATAGCTCTAAAAC (SEQ ID NO: 32)), wherein the single-stranded DNA serves as a transcription template.

In some embodiments, the gNA (e.g., gRNA) stem-loop sequence comprises the following RNA sequence: (5′>3′, GUUUUAGAGCUAUGCUGGAAACAGCAUAGCAAGUUAAAAUAAGGCUAGUCCG UUAUCAACUUGAAAAAGUGGCACCGAGUCGGUGCUUUUUUUC (SEQ ID NO: 33)).

In some embodiments, the CRISPR/Cas system protein is a Cpf1 protein. In some embodiments, the Cpf1 protein is isolated or derived from Franciscella species or Acidaminococcus species. In some embodiments, the gNA (e.g., gRNA) CRISPR/Cas system protein-binding sequence comprises the following RNA sequence: (5′>3′, AAUUUCUACUGUUGUAGAU (SEQ ID NO: 34)).

In some embodiments, the CRISPR/Cas system protein is a Cpf1 protein. In some embodiments, the Cpf1 protein is isolated or derived from Franciscella species or Acidaminococcus species. In some embodiments, a DNA sequence encoding the gNA (e.g., gRNA) CRISPR/Cas system protein-binding sequence comprises the following DNA sequence: (5′>3′, AATTTCTACTGTTGTAGAT (SEQ ID NO: 35)). In some embodiments, the DNA is single stranded. In some embodiments, the DNA is double stranded.

In some embodiments, provided herein is a gNA (e.g., gRNA) comprising a first NA segment comprising a targeting sequence and a second NA segment comprising a nucleic acid-guided nuclease (e.g., CRISPR/Cas) system protein-binding sequence. In some embodiments, the size of the first segment is 15 bp, 16 bp, 17 bp, 18 bp, 19 bp or 20 bp. In some embodiments, the second segment comprises a single segment, which comprises the gRNA stem-loop sequence. In some embodiments, the gRNA stem-loop sequence comprises the following RNA sequence: (5′>3′, GUUUUAGAGCUAGAAAUAGCAAGUUAAAAUAAGGCUAGUCCGUUAUCAACUU GAAAAAGUGGCACCGAGUCGGUGCUUUUUUU (SEQ ID NO: 30)). In some embodiments, the gRNA stem-loop sequence comprises the following RNA sequence: (5′>3′, GUUUUAGAGCUAUGCUGGAAACAGCAUAGCAAGUUAAAAUAAGGCUAGUCCG UUAUCAACUUGAAAAAGUGGCACCGAGUCGGUGCUUUUUUUC (SEQ ID NO: 33)). In some embodiments, the second segment comprises two sub-segments: a first RNA sub-segment (crRNA) that forms a hybrid with a second RNA sub-segment (tracrRNA), which together act to direct nucleic acid-guided nuclease (e.g., CRISPR/Cas) system protein binding. In some embodiments, the sequence of the second sub-segment comprises GUUUUAGAGCUAUGCUGUUUUG (SEQ ID NO: 36). In some embodiments, the first RNA segment and the second RNA segment together forms a crRNA sequence. In some embodiments, the other RNA that will form a hybrid with the second RNA segment is a tracrRNA. In some embodiments the tracrRNA comprises the sequence of 5′>3′, GGAACCAUUCAAAACAGCAUAGCAAGUUAAAAUAAGGCUAGUCCGUUAUCAA CUUGAAAAAGUGGCACCGAGUCGGUGCUUUUUUU (SEQ ID NO: 37).

In some embodiments, provided herein is a gNA (e.g., gRNA) comprising a first NA segment comprising a targeting sequence and a second NA segment comprising a nucleic acid-guided nuclease (e.g., CRISPR/Cas) system protein-binding sequence. In some embodiments, for example those embodiments wherein the CRISPR/Cas system protein is a Cpf1 system protein, the second segment is 5′ of the first segment. In some embodiments, the size of the first segment is 20 bp. In some embodiments, the size of the first segment is greater than 20 bp. In some embodiments, the size of the first segment is greater than 30 bp. In some embodiments, the second segment comprises a single segment, which comprises the gRNA stem-loop sequence. In some embodiments, the gRNA stem-loop sequence comprises the following RNA sequence: (5′>3′, AAUUUCUACUGUUGUAGAU (SEQ ID NO: 34)).

### CRISPR/Cas System Nucleic Acid-Guided Nucleases

In some embodiments, CRISPR/Cas system proteins are used in the embodiments provided herein. In some embodiments, CRISPR/Cas system proteins include proteins from CRISPR Type I systems, CRISPR Type II systems, and CRISPR Type III systems.

In some embodiments, CRISPR/Cas system proteins can be from any bacterial or archaeal species.

In some embodiments, the CRISPR/Cas system protein is isolated, recombinantly produced, or synthetic.

In some embodiments, the CRISPR/Cas system proteins are from, or are derived from CRISPR/Cas system proteins from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, Corynebacter diphtheria, Acidaminococcus, Lachnospiraceae bacterium or Prevotella.

In some embodiments, examples of CRISPR/Cas system proteins can be naturally occurring or engineered versions.

In some embodiments, naturally occurring CRISPR/Cas system proteins can belong to CAS Class I Type I, III, or IV, or CAS Class II Type II or V, and can include Cas9, Cas3, Cas8a-c, Cas10, CasX, CasY, Cas13, Cas14, Cse1, Csy 1, Csn2, Cas4, Csm2, Cmr5, Csf1, C2c2, and Cpf1.

In an exemplary embodiment, the CRISPR/Cas system protein comprises Cas9.

In an exemplary embodiment, the CRISPR/Cas system protein comprises Cpf1.

A “CRISPR/Cas system protein-gNA complex” refers to a complex comprising a CRISPR/Cas system protein and a guide NA (e.g. a gRNA or a gDNA). Where the gNA is a gRNA, the gRNA may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a gRNA) that contains crRNA and tracrRNA sequences. Alternatively, the guide RNA may be a single molecule (i.e. a gRNA) that comprises a crRNA sequence.

A CRISPR/Cas system protein may be at least 60% identical (e.g., at least 70%, at least 80%, or 90% identical, at least 95% identical or at least 98% identical or at least 99% identical) to a wild type CRISPR/Cas system protein. The CRISPR/Cas system protein may have all the functions of a wild type CRISPR/Cas system protein, or only one or some of the functions, including binding activity, nuclease activity, and nuclease activity.

The term “CRISPR/Cas system protein-associated guide NA” refers to a guide NA. The CRISPR/Cas system protein -associated guide NA may exist as isolated NA, or as part of a CRISPR/Cas system protein-gNA complex.

In some embodiments, the CRISPR/Cas system protein is an RNA-guided RNA nuclease (i.e., cuts RNA). Exemplary CRISPR/Cas system proteins that cut RNA include, but are not limited to C2c2. C2c2 (also known as Cas13a) is a class 2 type VI RNA-guided RNA-targeting CRISPR/Cas system protein. In some embodiments, the C2c2 nuclease is isolated or derived from Leptotrichia shahii. In some embodiments, C2c2 is guided by a single crRNA that cleaves an ssRNA carrying a complementary protospacer. An appropriate C2c2 crRNA sequence will be readily apparent to one of ordinary skill in the art.

In some embodiments, the CRISPR/Cas system protein is an RNA-guided DNA nuclease. In some embodiments, the DNA cleaved by the CRISPR/Cas system protein is double stranded. Exemplary RNA-guided DNA nucleases that cut double stranded DNA include, but are not limited to Cas9, Cpf1, CasX and CasY. Further exemplary RNA-guided DNA nucleases include Cas 10, Csm2, Csm3, Csm4, and Csm5. In some embodiments, Cas 10, Csm2, Csm3, Csm4, and Csm5 form a ribonucleoprotein complex with a gRNA.

In some embodiments, the RNA-guided DNA nuclease is CasX. In some embodiments, the CasX protein is dual guided (i.e., the gNA comprises a crRNA and a tracrRNA). In some embodiments, CasX recognizes a TTCN PAM located immediately 5′ of a sequence complementary to the targeting sequence. In some embodiments, the CasX protein is isolated or derived from Deltaproteobacteria or Planctomycetes. In some embodiments, the CasX protein is a CasX1, a CasX2 or a CasX3 protein. CasX proteins are described in WO/2018/064371, the contents of which are incorporated herein by reference in their entirety. Appropriate gNA sequences for CasX proteins will be readily apparent to the person of ordinary skill in the art.

In some embodiments, the RNA-guided DNA nuclease is CasY. In some embodiments, the CasY protein is dual guided (i.e., the gNA comprises a crRNA and a tracrRNA). In some embodiments, CasY recognizes a TA PAM located 5′ of the target sequence. CasY proteins are described in WO/2018/064352, the contents of which are incorporated herein by reference in their entirety. Appropriate gNA sequences for CasY proteins will be readily apparent to the person of ordinary skill in the art. In some embodiments, the CRISPR/Cas system protein is a RNA-guided DNA nuclease. In some embodiments, the DNA cleaved by the CRISPR/Cas system protein is single stranded. Exemplary RNA guided CRISPR/Cas system proteins that cut single stranded DNA include, but are not limited to Cas3 and Cas14. In some embodiments, the Cas14 protein does not require a PAM site.

### Cas9

In some embodiments, the CRISPR/Cas System protein nucleic acid-guided nuclease is or comprises Cas9. The Cas9 of the present disclosure can be isolated, recombinantly produced, or synthetic.

Examples of Cas9 proteins that can be used in the embodiments herein can be found in F. A. Ran, L. Cong, W. X. Yan, D. A. Scott, J. S. Gootenberg, A. J. Kriz, B. Zetsche, O. Shalem, X. Wu, K. S. Makarova, E. V. Koonin, P. A. Sharp, and F. Zhang; “In vivo genome editing using Staphylococcus aureus Cas9,” Nature 520, 186-191 (9 Apr. 2015) doi:10.1038/nature14299, which is incorporated herein by reference.

In some embodiments, the Cas9 is a Type II CRISPR system derived from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

In some embodiments, the Cas9 is a Type II CRISPR system derived from S. pyogenes and the PAM sequence is NGG located on the immediate 3′ end of the target specific guide sequence. The PAM sequences of Type II CRISPR systems from exemplary bacterial species can also include: Streptococcus pyogenes (NGG), Staph aureus (NNGRRT), Neisseria meningitidis (NNNNGATT), Streptococcus thermophiles (NNAGAA) and Treponema denticola (NAAAAC) which are all usable without deviating from the present disclosure.

In one exemplary embodiment, Cas9 sequence can be obtained, for example, from the pX330 plasmid (available from Addgene), re-amplified by PCR then cloned into pET30 (from EMD biosciences) to express in bacteria and purify the recombinant 6His tagged protein.

A “Cas9-gNA complex” refers to a complex comprising a Cas9 protein and a guide NA. A Cas9 protein may be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein may have all the functions of a wild type Cas9 protein, or only one or some of the functions, including binding activity, nuclease activity, and nuclease activity.

The term “Cas9-associated guide NA” refers to a guide NA as described above. The Cas9-associated guide NA may exist isolated, or as part of a Cas9-gNA complex. Non-CRISPR/Cas System Nucleic Acid-Guided Nucleases

In some embodiments, non-CRISPR/Cas system proteins are used in the embodiments provided herein.

In some embodiments, the non-CRISPR/Cas system proteins can be from any bacterial or archaeal species.

In some embodiments, the non-CRISPR/Cas system protein is isolated, recombinantly produced, or synthetic.

In some embodiments, the non-CRISPR/Cas system proteins are from, or are derived from Aquifex aeolicus, Therms thermophiles, Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, Natronobacterium gregoryi, or Corynebacter diphtheria.

In some embodiments, the non-CRISPR/Cas system proteins can be naturally occurring or engineered versions.

In some embodiments, a naturally occurring non-CRISPR/Cas system protein is NgAgo (Argonaute from Natronobacterium gregoryi).

A “non-CRISPR/Cas system protein-gNA complex” refers to a complex comprising a non-CRISPR/Cas system protein and a guide NA (e.g. a gRNA or a gDNA). Where the gNA is a gRNA, the gRNA may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a gRNA) that contains crRNA and tracrRNA sequences.

A non-CRISPR/Cas system protein may be at least 60% identical (e.g., at least 70%, at least 80%, or 90% identical, at least 95% identical or at least 98% identical or at least 99% identical) to a wild type non-CRISPR/Cas system protein. The non-CRISPR/Cas system protein may have all the functions of a wild type non-CRISPR/Cas system protein, or only one or some of the functions, including binding activity, nuclease activity, and nuclease activity.

The term “non-CRISPR/Cas system protein-associated guide NA” refers to a guide NA. The non-CRISPR/Cas system protein -associated guide NA may exist as isolated NA, or as part of a non-CRISPR/Cas system protein-gNA complex. Cpf1

In some embodiments, the CRISPR/Cas system protein nucleic acid-guided nuclease is or comprises a Cpf1 system protein. Cpf1 system proteins of the present disclosure can be isolated, recombinantly produced, or synthetic.

Cpf1 system proteins are Class II, Type V CRISPR system proteins. In some embodiments, the Cpf1 protein is isolated or derived from Francisella tularensis. In some embodiments, the Cpf1 protein is isolated or derived from Acidaminococcus, Lachnospiraceae bacterium or Prevotella.

Cpf1 system proteins bind to a single guide RNA comprising a nucleic acid-guided nuclease system protein-binding sequence (e.g., stem-loop) and a targeting sequence. The Cpf1 targeting sequence comprises a sequence located immediately 3′ of a Cpf1 PAM sequence in a target nucleic acid. Unlike Cas9, the Cpf1 nucleic acid-guided nuclease system protein-binding sequence is located 5′ of the targeting sequence in the Cpf1 gRNA. Cpf1 can also produce staggered rather than blunt ended cuts in a target nucleic acid. Following targeting of the Cpf1 protein-gRNA protein complex to a target nucleic acid, Francisella derived Cpf1, for example, cleaves the target nucleic acid in a staggered fashion, creating an approximately 5 nucleotide 5′ overhang 18-23 bases away from the PAM at the 3′ end of the targeting sequence. In contrast, cutting by a wild type Cas9 produces a blunt end 3 nucleotides upstream of the Cas9 PAM.

An exemplary Cpf1 gRNA stem-loop sequence comprises the following RNA sequence: (5′>3′, AAUUUCUACUGUUGUAGAU (SEQ ID NO: 34)).

A “Cpf1 protein-gNA complex” refers to a complex comprising a Cpf1 protein and a guide NA (e.g. a gRNA). Where the gNA is a gRNA, the gRNA may be composed of a single molecule, i.e., one RNA (“crRNA”) which hybridizes to a target and provides sequence specificity.

A Cpf1 protein may be at least 60% identical (e.g., at least 70%, at least 80%, or 90% identical, at least 95% identical or at least 98% identical or at least 99% identical) to a wild type Cpf1 protein. The Cpf1 protein may have all the functions of a wild type Cpf1 protein, or only one or some of the functions, including binding activity and nuclease activity.

Cpf1 system proteins recognize a variety of PAM sequences. Exemplary PAM sequences recognized by Cpf1 system proteins include, but are not limited to TTN, TCN and TGN. Additional Cpf1 PAM sequences include, but are not limited to TTTN. One feature of Cpf1 PAM sequences is that they have a higher A/T content than the NGG or NAG PAM sequences used by Cas9 proteins. Target nucleic acids, for example, different genomes, differ in their percent G/C content. For example, the genome of the human malaria parasite Plasmodium falciparum is known to be A/T rich. Alternatively, protein coding sequences within a genome frequently have a higher G/C content than the genome as a whole. The ratio of A/T to G/C nucleotides in a target genome affects the distribution and frequency of a given PAM sequence in that genome. For example, A/T rich genomes may have fewer NGG or NAG sequences, while G/C rich genomes may have fewer TTN sequences. Cpf1 system proteins expand the repertoire of PAM sequences available to the ordinarily skilled artisan, resulting superior flexibility and function of gRNA libraries.

### Catalytically Dead Nucleic Acid-Guided Nucleases

In some embodiments, engineered examples of nucleic acid-guided nuclease system (e.g., CRISPR/Cas system) proteins include catalytically dead nucleic acid-guided nuclease system proteins. The term “catalytically dead” generally refers to a nucleic acid-guided nuclease system protein that has inactivated nucleases (e.g., HNH and RuvC nucleases). Such a protein can bind to a target site in any nucleic acid (where the target site is determined by the guide NA), but the protein is unable to cleave or nick the target nucleic acid (e.g., double-stranded DNA). In some embodiments, the nucleic acid-guided nuclease system catalytically dead protein is a catalytically dead CRISPR/Cas system protein, such as catalytically dead Cas9 (dCas9). Accordingly, the dCas9 allows separation of the mixture into unbound nucleic acids and dCas9-bound fragments. In one embodiment, a dCas9/gRNA complex binds to targets determined by the gRNA sequence. The dCas9 bound can prevent cutting by Cas9 while other manipulations proceed. In another embodiment, the dCas9 can be fused to another enzyme, such as a transposase, to target that enzyme's activity to a specific site. Naturally occurring catalytically dead nucleic acid-guided nuclease system proteins can also be employed.

In another embodiment, the catalytically dead nucleic acid-guided nuclease can be fused to another enzyme, such as a transposase, to target that enzyme's activity to a specific site.

In some embodiments, the catalytically dead nucleic acid-guided nuclease is dCas9, dCpf1, dCas3, dCas8a-c, dCas10, dCse1, dCsy, dCsn2, dCas4, dCsm2, dCm5, dCsf1, dC2C2, dCasX, dCasY, dCas13, dCas14 or dNgAgo.

In one exemplary embodiment the catalytically dead nucleic acid-guided nuclease protein is a dCas9.

In one exemplary embodiment the catalytically dead nucleic acid-guided nuclease protein is a dCpf1.

### Nucleic Acid-Guided Nuclease Nickases

In some embodiments, engineered examples of nucleic acid-guided nucleases include nucleic acid-guided nuclease nickases (referred to interchangeably as nickase nucleic acid-guided nucleases).

In some embodiments, engineered examples of nucleic acid-guided nucleases include CRISPR/Cas system nickases or non-CRISPR/Cas system nickases, containing a single inactive catalytic domain.

In some embodiments, the nucleic acid-guided nuclease nickase is a Cas9 nickase, Cpf1 nickase, Cas3 nickase, Cas8a-c nickase, Cas10 nickase, Cse1 nickase, Csy1 nickase, Csn2 nickase, Cas4 nickase, Csm2 nickase, Cm5 nickase, Csf1 nickase, C2C2 nickase, a CasX nickase, a CasY nickase, a Cas 13 nickase, a Cas14 nickase or a NgAgo nickase.

In one embodiment, the nucleic acid-guided nuclease nickase is a Cas9 nickase.

In one embodiment, the nucleic acid-guided nuclease nickase is a Cpf1 nickase.

In some embodiments, a nucleic acid-guided nuclease nickase can be used to bind to target sequence. With only one active nuclease domain, the nucleic acid-guided nuclease nickase cuts only one strand of a target DNA, creating a single-strand break or “nick”. Depending on which mutant is used, the guide NA-hybridized strand or the non-hybridized strand may be cleaved. nucleic acid-guided nuclease nickases bound to 2 gNAs that target opposite strands can create a double-strand break in the nucleic acid. This “dual nickase” strategy increases the specificity of cutting because it requires that both nucleic acid-guided nuclease/gNA complexes be specifically bound at a site before a double-strand break is formed.

In exemplary embodiments, a Cas9 nickase can be used to bind to target sequence. The term “Cas9 nickase” refers to a modified version of the Cas9 protein, containing a single inactive catalytic domain, i.e., either the RuvC- or the HNH-domain. With only one active nuclease domain, the Cas9 nickase cuts only one strand of the target DNA, creating a single-strand break or “nick”. Depending on which mutant is used, the guide RNA-hybridized strand or the non-hybridized strand may be cleaved. Cas9 nickases bound to 2 gRNAs that target opposite strands will create a double-strand break in the DNA. This “dual nickase” strategy can increase the specificity of cutting because it requires that both Cas9/gRNA complexes be specifically bound at a site before a double-strand break is formed.

### Dissociable and Thermostable Nucleic Acid-Guided Nucleases

In some embodiments, thermostable nucleic acid-guided nucleases are used in the methods provided herein (thermostable CRISPR/Cas system nucleic acid-guided nucleases or thermostable non-CRISPR/Cas system nucleic acid-guided nucleases). In such embodiments, the reaction temperature is elevated, inducing dissociation of the protein; the reaction temperature is lowered, allowing for the generation of additional cleaved target sequences. In some embodiments, thermostable nucleic acid-guided nucleases maintain at least 50% activity, at least 55% activity, at least 60% activity, at least 65% activity, at least 70% activity, at least 75% activity, at least 80% activity, at least 85% activity, at least 90% activity, at least 95% activity, at least 96% activity, at least 97% activity, at least 98% activity, at least 99% activity, or 100% activity, when maintained for at least 75° C. for at least 1 minute. In some embodiments, thermostable nucleic acid-guided nucleases maintain at least 50% activity, when maintained for at least 1 minute at least at 75° C., at least at 80° C., at least at 85° C., at least at 90° C., at least at 91° C., at least at 92° C., at least at 93° C., at least at 94° C., at least at 95° C., 96° C., at least at 97° C., at least at 98° C., at least at 99° C., or at least at 100° C. In some embodiments, thermostable nucleic acid-guided nucleases maintain at least 50% activity, when maintained at least at 75° C. for at least 1 minute, 2 minutes, 3 minutes, 4 minutes, or 5 minutes. In some embodiments, a thermostable nucleic acid-guided nuclease maintains at least 50% activity when the temperature is elevated, lowered to 25° C-50° C. In some embodiments, the temperature is lowered to 25° C., to 30° C., to 35° C., to 40° C., to 45° C., or to 50° C. In one exemplary embodiment, a thermostable enzyme retains at least 90% activity after 1 min at 95° C.

In some embodiments, the thermostable nucleic acid-guided nuclease is thermostable Cas9, thermostable Cpf1, thermostable Cas3, thermostable Cas8a-c, thermostable Cas10, thermostable Cse1, thermostable Csy1, thermostable Csn2, thermostable Cas4, thermostable Csm2, thermostable Cm5, thermostable Csf1, thermostable C2C2, or thermostable NgAgo.

In some embodiments, the thermostable CRISPR/Cas system protein is thermostable Cas9.

Thermostable nucleic acid-guided nucleases can be isolated, for example, identified by sequence homology in the genome of thermophilic bacteria Streptococcus thermophilus and Pyrococcus furiosus. Nucleic acid-guided nuclease genes can then be cloned into an expression vector. In one exemplary embodiment, a thermostable Cas9 protein is isolated.

In another embodiment, a thermostable nucleic acid-guided nuclease can be obtained by in vitro evolution of a non-thermostable nucleic acid-guided nuclease. The sequence of a nucleic acid-guided nuclease can be mutagenized to improve its thermostability.

### Kits and Articles of Manufacture

The present disclosure provides kits comprising any one or more of the compositions described herein, not limited to adapters, gNAs (e.g., gRNAs or gDNAs), gNA collections (e.g., gRNA or gDNA pluralities), modification-sensitive restriction enzymes, controls and the like.

In one exemplary embodiment, the kit comprises of gRNAs wherein the gRNAs are targeted to human genomic or other sources of DNA sequences.

The present disclosure also provides all essential reagents and instructions for carrying out the methods of enriching a sample for nucleic acids of interest using differences in nucleotide modification, as described herein.

Also provided herein is computer software monitoring the information before and after enriching a sample using the methods provided herein. In one exemplary embodiment, the software can compute and report the abundance of sequences of nucleic acids targeted for depletion in the sample before and after applying the methods described herein, to assess the level of off-target depletion, and wherein the software can check the efficacy of targeted-depletion/encrichment/capture/partitioning/labeling/regulation/editing by comparing the abundance of the sequence of interest before and after processing the sample using the methods of enrichment provided herein.

All publications mentioned in the above specification are herein incorporated by reference. Various modifications and variations of the described products, systems, uses, processes and methods of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure, which are obvious to those skilled in molecular biology and biotechnology or related fields, are intended to be within the scope of the following claims.

### ENUMERATED EMBODIMENTS

The invention may be defined by reference to the following enumerated, illustrative embodiments:

1. A method of enriching a sample for nucleic acids of interest relative to nucleic acids targeted for depletion by about at least about 2-fold, comprising using differences in nucleotide modification between the nucleic acids of interest and the nucleic acids targeted for depletion.

2. A method of enriching a sample for nucleic acids of interest relative to nucleic acids targeted for depletion by about at least about 2-fold, comprising using differences in nucleotide modification between the nucleic acids of interest and the nucleic acids targeted for depletion, and not comprising size selection or modification-sensitive targeted binding.

3. A method of enriching a sample for nucleic acids of interest relative to nucleic acids targeted for depletion by about at least about 2-fold, comprising using differences in nucleotide modification between the nucleic acids of interest and the nucleic acids targeted for depletion to ligate adapters to the nucleic acids of interest and not to the nucleic acids targeted for depletion.

4. A method of enriching a sample for nucleic acids of interest comprising:
a. providing a sample comprising nucleic acids of interest and nucleic acids targeted for depletion, wherein at least a subset of the nucleic acids of interest or a subset of the nucleic acids targeted for depletion comprise a plurality of first recognition sites for a first modification-sensitive restriction enzyme;
b. terminally dephosphorylating a plurality of the nucleic acids in the sample;
c. contacting the sample from (b) with the first modification-sensitive restriction enzyme under conditions that allow for cleavage of at least some of the first modification-sensitive restriction sites in the nucleic acids in the sample; and
d. contacting the sample from (c) with adapters under conditions that allow for the ligation of the adapters to a 5′ and 3′ end of a plurality of the nucleic acids of interest;
   thereby generating a sample enriched for nucleic acids of interest that are adapter-ligated on their 5′ and 3′ ends.

5. The method of embodiment 4, wherein the nucleic acids of interest and the nucleic acids targeted for depletion are fragmented prior to (a).

6. The method of embodiment 4 or 5, wherein both the nucleic acids of interest and the nucleic acids targeted for depletion each comprise a plurality of first recognition sites for the first modification-sensitive restriction enzyme.

7. The method of embodiment 6, wherein a frequency of nucleotide modification within or adjacent to the plurality of first recognitions sites is not the same in nucleic acids of interest as in the nucleic acids targeted for depletion.

8. The method of any one of embodiments 4-7, wherein activity of the first modification-sensitive restriction enzyme is blocked by modification of a nucleotide within or adjacent to its cognate recognition site.

9. The method of embodiment 8, wherein the plurality of first recognition sites in the nucleic acids targeted for depletion are modified more frequently than the plurality of first recognition sites in the nucleic acids of interest.

10. The method of embodiment 8 or 9, wherein the first modification-sensitive restriction enzyme comprises a restriction enzyme selected from the group consisting of AatII, AccII, Aor13HI, Aor51HI, BspT104I, BssHII, Cfr10I, ClaI, CpoI, Eco52I, HaeII, HapII, HhaI , MluI, NaeI, NotI, NruI, NsbI, PmaCI, Psp1406I, PvuI, SacII, SalI, SmaI, SnaBI, AluI and Sau3AI.

11. The method of embodiment 8 or 9, wherein the first modification-sensitive restriction enzyme is comprises a restriction enzyme selected from the group consisting of AluI and Sau3AI.

12. The method of embodiment 4-7, wherein the first modification-sensitive restriction enzyme is active at a recognition site comprising at least one modified nucleotide and is not active at a recognition site that does not comprise at least one modified nucleotide.

13. The method of embodiment 12, wherein the plurality of first recognition sites in the nucleic acids targeted for depletion are modified more frequently than the plurality of first recognition sites in the nucleic acids of interest.

14. The method of embodiment 12 or 13, wherein the first modification-sensitive restriction enzyme comprises a restriction enzyme selected from the group consisting of AbaSI, FspEI, LpnPI, MspII or McrBC.

15. The method of any one of embodiments 12-13, wherein the modification comprises 5-hydroxymethylcytosine.

16. The method of embodiment 15, wherein the first modification-sensitive restriction enzyme comprises AbaSI and the method further comprises contacting the sample with T4 phage β-glucosyltransferase prior to step (c).

17. The method of any one of embodiments 12-14, wherein the modification comprises glucosylhydroxymethylcytosine.

18. The method of embodiment 17, wherein the first modification-sensitive restriction enzyme comprises AbaSI.

19. The method of any one of embodiments 12-14, wherein the modification comprises methylcytosine.

20. The method of embodiment 19, wherein the first modification-sensitive restriction enzyme comprises McrBC.

21. The method of any one of embodiments 12-20, wherein the nucleic acids of interest comprise at least one DpnI recognition site, and wherein the method further comprises, prior to step (c), contacting the sample with DpnI and T4 polymerase.

22. The method of embodiment 21, wherein the T4 polymerase replaces methylated A and C nucleotides with unmethylated A and C nucleotides within or adjacent to the at least one DpnI recognition site.

23. The method of any one of embodiments 12-22, further comprising, prior to step (d), contacting the sample from (c) with an exonuclease under conditions that allow for the successive removal of nucleotides from a phosphorylated end of a nucleic acid.

24. The method of embodiment 23, wherein the exonuclease comprises a Lambda nuclease, Exonuclease III or BAL-31.

25. The method of any one of embodiments 4-24, wherein terminally dephosphorylating the nucleic acids in the sample in step (b) comprises a phosphatase.

26. The method of embodiment 25, wherein the phosphatase is an alkaline phosphatase.

27. The method of embodiment 26, wherein the alkaline phosphatase is a shrimp alkaline phosphatase.

28. The method of any one of embodiments 4-27, further comprising:
e. contacting the adapter-ligated nucleic acids from (d) with a second modification-sensitive restriction enzyme under conditions that allow the second modification-sensitive restriction enzyme to cut a second recognition site,
   wherein at least a subset of the nucleic acids targeted for depletion comprise a plurality of second recognition sites for a second modification-sensitive restriction enzyme, and
   wherein the second modification-sensitive restriction enzyme targets recognition sites comprising at least one modified nucleotide and does not target recognition sites that do not comprise at least one modified nucleotide,
   thereby generating a collection of nucleic acids targeted for depletion that are adapter-ligated on one end and a collection of nucleic acids of interest that are adapter-ligated on both ends.

29. The method of embodiment 28, wherein the nucleic acids of interest and the nucleic acids targeted for depletion each comprise a plurality of second recognition sites for the second modification-sensitive restriction enzyme.

30. The method of embodiment 29, wherein the plurality of second recognition sites in the nucleic acids targeted for depletion are modified more frequently than the plurality of second recognition sites in the nucleic acids of interest.

31. The method of any one of embodiments 4-30, further comprising contacting the sample after step (d) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends.

32. The method of embodiment 31, wherein the method comprises contacting the sample with at least 10² unique nucleic acid-guided nuclease-gNA complexes, at least 10³ unique nucleic acid-guided nuclease-gNA complexes, 10⁴ unique nucleic acid-guided nuclease-gNA complexes or 10⁵ unique nucleic acid-guided nuclease-gNA complexes.

33. The method of embodiment 31 or 32, wherein the nucleic acid-guided nuclease is Cas9, Cpf1, Cas3, Cas8a-c, Cas10, Cse1, Csy, Csn2, Cas4, Csm2, CasX, CasY, Cas13, Cas14 or Cm5.

34. The method of embodiment 31 or 32, wherein the nucleic acid-guided nuclease is Cas9, Cpf1 or a combination thereof.

35. The method of any one of embodiments 31-34, wherein the nucleic acid-guided nuclease is a Cas9 or Cpf1 nickase.

36. The method of any one of embodiments 31-35, wherein the nucleic acid-guided nuclease is thermostable.

37. The method of any one of embodiments 31-36, wherein the gNA is a deoxyribonucleic acid (DNA) or a ribonucleic acid (RNA).

38. The method of any one of embodiments 4-37, further comprising amplifying, sequencing or cloning the nucleic acids of interest that are adapter-ligated on their 5′ and 3′ ends using the adapters.

39. The method of any one of embodiments 1-38, wherein the nucleotide modification comprises adenine modification or cytosine modification.

40. The method of embodiment 39, wherein the adenine modification comprises adenine methylation.

41. The method of embodiment 40, wherein the adenine methylation comprises Dam methylation or EcoKI methylation.

42. The method of embodiment 39, wherein the cytosine modification comprises 5-methylcytosine, 5-hydroxymethlcytosine, 5-formylcytosine, 5-carboxylcytosine, 5-glucosylhydroxymethylcytosine or 3-methylcytosine.

43. The method of embodiment 39, wherein the cytosine modification comprises cytosine methylation.

44. The method of embodiment 43, wherein the cytosine methylation comprises CpG methylation, CpA methylation, CpT methylation, CpC methylation or a combination thereof.

45. The method of embodiment 43, wherein the cytosine methylation comprises Dcm methylation, DNMT1 methylation, DNMT3A methylation or DNMT3B methylation.

46. The method of any one of embodiments 28-45, wherein the second modification-sensitive restriction enzyme comprises a restriction enzyme selected from the group consisting of AbaSI, FspEI, LpnPI, MspJI or McrBC.

47. The method of any one of embodiments 28-38, wherein the modification comprises 5-hydroxymethylcytosine.

48. The method of embodiment 47, wherein and the second modification-sensitive restriction enzyme comprises AbaSI and the method further comprises contacting the sample with T4 phage β-glucosyltransferase prior to step (e).

49. The method of any one of embodiments 28-38, wherein the modification comprises glucosylhydroxymethylcytosine.

50. The method of embodiment 49, wherein the second modification-sensitive restriction enzyme comprises AbaSI.

51. The method of any one of embodiments 28-38, wherein the modification comprises methylcytosine.

52. The method of embodiment 51, wherein the second modification-sensitive restriction enzyme comprises McrBC.

53. The method of any one of embodiments 28-52, wherein the nucleic acids of interest comprise at least one DpnI recognition site, and wherein the method further comprises, prior to step (e), contacting the sample with DpnI and T4 polymerase.

54. The method of embodiment 53, wherein the T4 polymerase replaces u methylated A and C nucleotides with unmethylated A and C nucleotides within or adjacent to the at least one DpnI recognition site.

55. The method of any one of embodiments 1-54, wherein the nucleic acids targeted for depletion comprise host nucleic acids and the nucleic acids of interest comprise non-host nucleic acids.

56. The method of embodiment 55, wherein the non-host comprises a bacterium, a fungus or a virus.

57. The method of embodiment 55, wherein the non-host comprises multiple species of organisms.

58. The method of embodiment 55, wherein the host is a mammal, a bird, a reptile or an insect.

59. The method of embodiment 58, wherein the mammal is a human, cow, horse, sheep, pig, monkey, dog, cat, rat, rabbit, mouse or gerbil.

60. The method of any one of embodiments 1-59, wherein the nucleic acids targeted for depletion comprise transcriptionally active sites and the nucleic acids of interest comprise repetitive sequences.

61. The method of any one of embodiments 4-60, wherein the adapter-ligated nucleic acids of interest and nucleic acids targeted for depletion range from 50-1000 bp.

62. The method of any one of embodiments 1-61, wherein the nucleic acids of interest comprise less than 50% of the total nucleic acids in the sample.

63. The method of any one of embodiments 1-61, wherein the nucleic acids of interest comprise less than 30% of the total nucleic acids in the sample.

64. The method of any one of embodiments 1-61, wherein the nucleic acids of interest comprise less than 5% of the total nucleic acids in the sample.

65. The method of any one of embodiments 1-64, wherein the sample is any one of a biological sample, a clinical sample, a forensic sample or an environmental sample.

66. The method of any one of embodiments 1-64, wherein the sample is selected from whole blood, plasma, serum, tears, saliva, mucous, cerebrospinal fluid, teeth, bone, fingernails, feces, urine, tissue, and a biopsy.

67. A method of enriching a sample for nucleic acids of interest comprising:
a. providing a sample comprising nucleic acids of interest and nucleic acids targeted for depletion, wherein at least a subset of the nucleic acids targeted for depletion comprise a plurality of recognition sites for a modification-sensitive restriction enzyme;
b. terminally dephosphorylating a plurality of the nucleic acids in the sample;
c. contacting the sample from (b) with the modification-sensitive restriction enzyme under conditions that allow for the cleavage of the modification-sensitive restriction sites in the nucleic acids in the sample, thereby generating nucleic acids with exposed terminal phosphates; and
d. contacting the sample with an exonuclease under conditions that allow for the successive removal of nucleotides from a phosphorylated end of a nucleic acid; thereby generating a sample enriched for nucleic acids of interest.

68. The method of embodiment 67, wherein the nucleic acids of interest and the nucleic acids targeted for depletion are fragmented prior to step (a).

69. The method of embodiment 67 or 68, wherein the nucleic acids of interest and the nucleic acids targeted for depletion each comprise a plurality of recognition sites for the modification-sensitive restriction enzyme.

70. The method of embodiment 69, wherein the plurality of recognition sites in the nucleic acids targeted for depletion are modified more frequently than the plurality of recognition sites in the nucleic acids of interest.

71. The method of any one of embodiments 67-70, wherein the nucleic acids of interest comprise at least one DpnI recognition site, and wherein the method further comprises, prior to step (c), contacting the sample with DpnI and T4 polymerase.

72. The method of embodiment 71, wherein the T4 polymerase replaces methylated A and C nucleotides with unmethylated A and C nucleotides within or adjacent to the at least one DpnI recognition site.

73. The method of any one of embodiments 67-72, wherein the modification comprises adenine modification or cytosine modification.

74. The method of embodiment 73, wherein the adenine modification comprises adenine methylation.

75. The method of embodiment 73, wherein the adenine methylation comprises Dam methylation or EcoKI methylation.

76. The method of embodiment 73, wherein the cytosine modification comprises 5-methylcytosine, 5-hydroxymethlcytosine, 5-formylcytosine, 5-carboxylcytosine, 5-glucosylhydroxymethylcytosine or 3-methylcytosine.

77. The method of embodiment 73, wherein the cytosine modification comprises cytosine methylation.

78. The method of embodiment 77, wherein the cytosine methylation comprises CpG methylation, CpA methylation, CpT methylation, CpC methylation or a combination thereof.

79. The method of embodiment 73, wherein the cytosine methylation comprises Dcm methylation, DNMT1 methylation, DNMT3A methylation or DNMT3B methylation.

80. The method of any one of embodiments 67-79, wherein the modification-sensitive restriction enzyme comprises a restriction enzyme selected from the group consisting of AbaSI, FspEI, LpnPI, MspII or McrBC.

81. The method of any one of embodiments 67-72, wherein the modification comprises 5-hydroxymethylcytosine.

82. The method of embodiment 81, wherein the modification-sensitive restriction enzyme comprises AbaSI and the method further comprises contacting the sample with T4 phage β-glucosyltransferase prior to step (c).

83. The method of any one of embodiments 67-72, wherein the modification comprises glucosylhydroxymethylcytosine.

84. The method of embodiment 83, wherein the modification-sensitive restriction enzyme comprises AbaSI.

85. The method of any one of embodiments 67-72, wherein the modification comprises methylcytosine.

86. The method of embodiment 85, wherein the modification-sensitive restriction enzyme comprises McrBC.

87. The method of embodiment 67-86, wherein the exonuclease is a Lambda nuclease, Exonuclease III or BAL-31.

88. The method of any one of embodiments 67-87, wherein the terminally dephosphorylating the nucleic acids in the sample in step (b) comprises a phosphatase.

89. The method of embodiment 88, wherein the phosphatase is an alkaline phosphatase.

90. The method of embodiment 74, wherein the alkaline phosphatase is a shrimp alkaline phosphatase.

91. The method of any one of embodiments 67-90, further comprising:
e. contacting the sample from (d) with adapters under conditions that allow for the ligation of the adapters to a 5′ and 3′ end of a plurality of the nucleic acids of interest;
   thereby generating a sample enriched for nucleic acids of interest that are adapter-ligated on their 5′ and 3′ ends.

92. The method of any one of embodiments 67-91, further comprising contacting the sample after step (d) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends.

93. The method of embodiment 92, wherein the method comprises contacting the sample with at least 10² unique nucleic acid-guided nuclease-gNA complexes, at least 10³ unique nucleic acid-guided nuclease-gNA complexes, 10⁴ unique nucleic acid-guided nuclease-gNA complexes or 10⁵ unique nucleic acid-guided nuclease-gNA complexes.

94. The method of embodiment 92 or 93, wherein the nucleic acid-guided nuclease is Cas9, Cpf1, Cas3, Cas8a-c, Cas10, Cse1, Csy1, Csn2, Cas4, Csm2, CasX, CasY, Cas13, Cas14 or Cm5.

95. The method of embodiment 92 or 93, wherein the nucleic acid-guided nuclease is Cas9, Cpf1 or a combination thereof.

96. The method of any one of embodiments 92-95, wherein the nucleic acid-guided nuclease is a Cas9 or Cpf1 nickase.

97. The method of any one of embodiments 92-96, wherein the nucleic acid-guided nuclease is thermostable.

98. The method of any one of embodiments 92-97, wherein the gNA is a deoxyribonucleic acid (DNA) or a ribonucleic acid (RNA).

99. The method of any one of embodiments 67-98, further comprising amplifying, sequencing or cloning the nucleic acids of interest that are adapter-ligated on their 5′ and 3′ ends using the adapters.

100. The method of any one of embodiments 67-99, wherein the nucleic acids targeted for depletion comprise host nucleic acids and the nucleic acids of interest comprise non-host nucleic acids.

101. The method of embodiment 100, wherein the non-host comprises a bacterium, a fungus or a virus.

102. The method of embodiment 100, wherein the non-host comprises multiple species of organisms.

103. The method of embodiment 100, wherein the host is a mammal, a bird, a reptile or an insect.

104. The method of embodiment 103, wherein the mammal is a human, cow, horse, sheep, pig, monkey, dog, cat, rat, rabbit, mouse or gerbil.

105. The method of any one of embodiments 67-104, wherein the nucleic acids targeted for depletion comprise transcriptionally active sites and the nucleic acids of interest comprise repetitive sequences.

106. The method of any one of embodiments 67-105, wherein the adapter-ligated nucleic acids of interest and nucleic acids targeted for depletion range from 50-1000 bp.

107. The method of any one of embodiments 67-106, wherein the nucleic acids of interest comprise less than 50% of the total nucleic acids in the sample.

108. The method of any one of embodiments 67-106, wherein the nucleic acids of interest comprise less than 30% of the total nucleic acids in the sample.

109. The method of any one of embodiments 67-106, wherein the nucleic acids of interest comprise less than 5% of the total nucleic acids in the sample.

110. The method of any one of embodiments 67-106, wherein the sample is any one of a biological sample, a clinical sample, a forensic sample or an environmental sample.

111. The method of any one of embodiments 67-106, wherein the sample is selected from whole blood, plasma, serum, tears, saliva, mucous, cerebrospinal fluid, teeth, bone, fingernails, feces, urine, tissue, and a biopsy

112. A method of enriching a sample for nucleic acids of interest comprising:
a. providing a sample comprising nucleic acids of interest and nucleic acids targeted for depletion, wherein at least a subset of the nucleic acids targeted for depletion comprise a plurality of recognition sites for a modification-sensitive restriction enzyme;
b. contacting the sample with adapters under conditions that allow for the ligation of the adapters to a 5′ and 3′ end of a plurality of the nucleic acids in the sample; and
c. contacting the sample from (b) with the modification-sensitive restriction enzyme under conditions that allow for the cleavage of the modification-sensitive restriction sites in the nucleic acids in the sample;
   thereby generating a sample enriched for nucleic acids of interest that are adapter-ligated on their 5′ and 3′ ends.

113. The method of embodiment 112, wherein the nucleic acids of interest and the nucleic acids targeted for depletion are fragmented prior to step (a).

114. The method of embodiment 112 or 113, wherein both the nucleic acids of interest and the nucleic acids targeted for depletion each comprise a plurality of recognition sites for the modification-sensitive restriction enzyme.

115. The method of any one of embodiments 112-114, wherein the plurality of recognition sites in the nucleic acids targeted for depletion are modified more frequently than the plurality of recognition sites in the nucleic acids of interest.

116. The method of any one of embodiments 112-115, wherein the nucleic acids of interest comprise at least one DpnI recognition site, and wherein the method further comprises, prior to step (c), contacting the sample with DpnI and T4 polymerase.

117. The method of embodiment 116, wherein the T4 polymerase replaces methylated A and C nucleotides with unmethylated A and C nucleotides within or adjacent to the at least one DpnI recognition site.

118. The method of any one of embodiments 112-117, wherein the modification comprises adenine modification or cytosine modification.

119. The method of embodiment 118, wherein the adenine modification comprises adenine methylation.

120. The method of embodiment 119, wherein the adenine methylation comprises Dam methylation or EcoKI methylation.

121. The method of embodiment 118, wherein the cytosine modification comprises 5-methylcytosine, 5-hydroxymethlcytosine, 5-formylcytosine, 5-carboxylcytosine, 5-glucosylhydroxymethylcytosine or 3-methylcytosine.

122. The method of embodiment 118, wherein the cytosine modification comprises cytosine methylation.

123. The method of embodiment 122, wherein the cytosine methylation comprises CpG methylation, CpA methylation, CpT methylation, CpC methylation or a combination thereof.

124. The method of embodiment 122, wherein the cytosine methylation comprises Dcm methylation, DNMT1 methylation, DNMT3A methylation or DNMT3B methylation.

125. The method of any one of embodiments 112-124, wherein the modification-sensitive restriction enzyme comprises AbaSI, FspEI, LpnPI, MspJI or McrBC.

126. The method of any one of embodiments 112-117, wherein the modification comprises 5-hydroxymethylcytosine.

127. The method of embodiment 126, wherein and the modification-sensitive restriction enzyme comprises AbaSI the method further comprises contacting the sample with T4 phage β-glucosyltransferase prior to (c).

128. The method of any one of embodiments 112-117, wherein the modification comprises glucosylhydroxymethylcytosine.

129. The method of embodiment 128, wherein the modification-sensitive restriction enzyme comprises AbaSI.

130. The method of any one of embodiments 112-117, wherein the modification comprises methylcytosine.

131. The method of embodiment 130, wherein the modification-sensitive restriction enzyme comprises McrBC.

132. The method of any one of embodiments 112-131, further comprising contacting the sample after step (c) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends.

133. The method of embodiment 132, wherein the method comprises contacting the sample with at least 10² unique nucleic acid-guided nuclease-gNA complexes, at least 10³ unique nucleic acid-guided nuclease-gNA complexes, 10⁴ unique nucleic acid-guided nuclease-gNA complexes or 10⁵ unique nucleic acid-guided nuclease-gNA complexes.

134. The method of embodiment 132 or 133, wherein the nucleic acid-guided nuclease is Cas9, Cpf1, Cas3, Cas8a-c, Cas10, Cse1, Csy1, Csn2, Cas4, Csm2, CasX, CasY, Cas13, Cas14 or Cm5.

135. The method of embodiment 132 or 133, wherein the nucleic acid-guided nuclease is Cas9, Cpf1 or a combination thereof.

136. The method of any one of embodiments 132-135, wherein the nucleic acid-guided nuclease is a Cas9 or Cpf1 nickase.

137. The method of any one of embodiments 132-136, wherein the nucleic acid-guided nuclease is thermostable.

138. The method of any one of embodiments 112-137, wherein the gNA is a deoxyribonucleic acid (DNA) or a ribonucleic acid (RNA).

139. The method of any one of embodiments 112-138, further comprising amplifying, sequencing or cloning the nucleic acids of interest that are adapter-ligated on their 5′ and 3′ ends using the adapters.

140. The method of any one of embodiments 112-139, wherein the nucleic acids targeted for depletion comprise host nucleic acids and the nucleic acids of interest comprise non-host nucleic acids.

141. The method of embodiment 140, wherein the non-host comprises a bacterium, a fungus or a virus.

142. The method of embodiment 140, wherein the non-host comprises multiple species of organisms.

143. The method of embodiment 140, wherein the host is a mammal, a bird, a reptile or an insect.

144. The method of embodiment 143, wherein the mammal is a human, cow, horse, sheep, pig, monkey, dog, cat, rat, rabbit, mouse or gerbil.

145. The method of any one of embodiments 112-144, wherein the nucleic acids targeted for depletion comprise transcriptionally active sites and the nucleic acids of interest comprise repetitive sequences.

146. The method of any one of embodiments 112-145, wherein the adapter-ligated nucleic acids of interest and nucleic acids targeted for depletion range from 50-1000 bp.

147. The method of any one of embodiments 112-146, wherein the nucleic acids of interest comprise less than 50% of the total nucleic acids in the sample.

148. The method of any one of embodiments 112-146, wherein the nucleic acids of interest comprise less than 30% of the total nucleic acids in the sample.

149. The method of any one of embodiments 112-146, wherein the nucleic acids of interest comprise less than 5% of the total nucleic acids in the sample.

150. The method of any one of embodiments 112-149, wherein the sample is any one of a biological sample, a clinical sample, a forensic sample or an environmental sample.

151. The method of any one of embodiments 112-149, wherein the sample is selected from whole blood, plasma, serum, tears, saliva, mucous, cerebrospinal fluid, teeth, bone, fingernails, feces, urine, tissue, and a biopsy.

152. A method of enriching a sample for nucleic acids of interest comprising:
a. providing a sample comprising nucleic acids of interest and nucleic acids targeted for depletion,
   wherein at least a subset of the nucleic acids of interest or a subset of the nucleic acids targeted for depletion comprise a plurality of first recognition sites for a first modification-sensitive restriction enzyme, and
   wherein activity of the first modification-sensitive restriction enzyme is blocked by modification of a nucleotide within or adjacent to its cognate recognition site;
b. terminally dephosphorylating a plurality of the nucleic acids in the sample;
c. contacting the sample from (b) with the first modification-sensitive restriction enzyme under conditions that allow for cleavage of at least some of the first modification-sensitive restriction sites in the nucleic acids in the sample; and
d. contacting the sample from (c) with adapters under conditions that allow for the ligation of the adapters to a 5′ and 3′ end of a plurality of the nucleic acids of interest;
   thereby generating a sample enriched for nucleic acids of interest that are adapter-ligated on their 5′ and 3′ ends.

153. The method of embodiment 152, wherein the nucleic acids of interest and the nucleic acids targeted for depletion are fragmented prior to (a).

154. The method of embodiment 152 or 153, wherein both the nucleic acids of interest and the nucleic acids targeted for depletion each comprise a plurality of first recognition sites for the first modification-sensitive restriction enzyme.

155. The method of embodiment 154, wherein a frequency of nucleotide modification within or adjacent to the plurality of first recognitions sites is not the same in nucleic acids of interest as in the nucleic acids targeted for depletion.

156. The method of embodiment 155, wherein the plurality of first recognition sites in the nucleic acids targeted for depletion are modified more frequently than the plurality of first recognition sites in the nucleic acids of interest.

157. The method of embodiment 155 or 156, wherein the first modification-sensitive restriction enzyme comprises a restriction enzyme selected from the group consisting of AatII, AccII, Aor13HI, Aor51HI, BspT104I, BssHII, Cfr10I, ClaI, CpoI, Eco52I, HaeII, HapII, HhaI , MluI, NaeI, NotI, NruI, NsbI, PmaCI, Psp14061, PvuI, SacII, SalI, SmaI, SnaBI, AluI and Sau3AI.

158. The method of embodiment 155 or 156, wherein the first modification-sensitive restriction enzyme is comprises a restriction enzyme selected from the group consisting of AluI and Sau3AI.

What is claimed is:

1. A method of enriching a sample for nucleic acids of interest comprising:
a. providing a sample comprising nucleic acids of interest and nucleic acids targeted for depletion, wherein at least a subset of the nucleic acids of interest or a subset of the nucleic acids targeted for depletion comprise a plurality of first recognition sites for a first modification-sensitive restriction enzyme;
b. terminally dephosphorylating a plurality of the nucleic acids in the sample;
c. contacting the sample from (b) with the first modification-sensitive restriction enzyme under conditions that allow for cleavage of at least some of the first modification-sensitive restriction sites in the nucleic acids in the sample; and
d. contacting the sample from (c) with adapters under conditions that allow for the ligation of the adapters to a 5′ and 3′ end of a plurality of the nucleic acids of interest;
thereby generating a sample enriched for nucleic acids of interest that are adapter-ligated on their 5′ and 3′ ends.

2. The method of claim 1, wherein both the nucleic acids of interest and the nucleic acids targeted for depletion comprise a plurality of first recognition sites for the first modification-sensitive restriction enzyme.

3. The method of claim 2, wherein a frequency of nucleotide modification within or adjacent to the plurality of first recognitions sites is not the same in nucleic acids of interest as in the nucleic acids targeted for depletion.

4. The method of any one of claims 1-3, wherein activity of the first modification-sensitive restriction enzyme is blocked by modification of a nucleotide within or adjacent to its cognate recognition site.

5. The method of claim 4, wherein the plurality of first recognition sites in the nucleic acids targeted for depletion are modified more frequently than the plurality of first recognition sites in the nucleic acids of interest.

6. The method of claim 4 or 5, wherein the first modification-sensitive restriction enzyme comprises a restriction enzyme selected from the group consisting of AatII, AccII, Aor13HI, Aor51HI, BspT104I, BssHII, Cfr101I, ClaI, CpoI, Eco52I, HaeII, HapII, HhaI , MluI, NaeI, NotI, NruI, NsbI, PmaCI, Psp14061, PvuI, SacII, SalI, SmaI, SnaBI, AluI and Sau3AI.

7. The method of claim 4 or 5, wherein the first modification-sensitive restriction enzyme is comprises a restriction enzyme selected from the group consisting of AluI and Sau3AI.

8. The method of claim 1-3, wherein the first modification-sensitive restriction enzyme is active at a recognition site comprising at least one modified nucleotide and is not active at a recognition site that does not comprise at least one modified nucleotide.

9. The method of claim 8, wherein the plurality of first recognition sites in the nucleic acids targeted for depletion are modified more frequently than the plurality of first recognition sites in the nucleic acids of interest.

10. The method of claim 8 or 9, wherein the first modification-sensitive restriction enzyme comprises a restriction enzyme selected from the group consisting of AbaSI, FspEI, LpnPI, MspJI or McrBC.

11. The method of claim 8 or 9, wherein the modification comprises 5-hydroxymethylcytosine,
the first modification-sensitive restriction enzyme comprises AbaSI, and the method further comprises contacting the sample with T4 phage β-glucosyltransferase prior to step (c).

12. The method of claim 8 or 9, wherein the modification comprises glucosylhydroxymethylcytosine, and the first modification-sensitive restriction enzyme comprises AbaSI.

13. The method of claim 8 or 9, wherein the modification comprises methylcytosine, and the first modification-sensitive restriction enzyme comprises McrBC.

14. The method of any one of claims 8-13, wherein the nucleic acids of interest comprise at least one DpnI recognition site, and wherein the method further comprises, prior to step (c), contacting the sample with DpnI and T4 polymerase thereby replacing methylated A and C nucleotides with unmethylated A and C nucleotides within or adjacent to the at least one DpnI recognition site.

15. The method of any one of claims 8-14, further comprising, prior to step (d), contacting the sample from (c) with an exonuclease under conditions that allow for the successive removal of nucleotides from a phosphorylated end of a nucleic acid.

16. The method of any one of claims 1-15, further comprising:
e. contacting the adapter-ligated nucleic acids from (d) with a second modification-sensitive restriction enzyme under conditions that allow the second modification-sensitive restriction enzyme to cut a second recognition site,
wherein at least a subset of the nucleic acids targeted for depletion comprise a plurality of second recognition sites for a second modification-sensitive restriction enzyme, and
wherein the second modification-sensitive restriction enzyme targets recognition sites comprising at least one modified nucleotide and does not target recognition sites that do not comprise at least one modified nucleotide,
thereby generating a collection of nucleic acids targeted for depletion that are adapter-ligated on one end and a collection of nucleic acids of interest that are adapter-ligated on both ends.

17. The method of any one of claims 1-16, further comprising contacting the sample after step (d) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends.

18. The method of any one of claims 1-17, further comprising amplifying, sequencing or cloning the nucleic acids of interest that are adapter-ligated on their 5′ and 3′ ends using the adapters.

19. The method of any one of claims 1-18, wherein the nucleotide modification comprises adenine modification or cytosine modification.

20. The method of claim 19, wherein the adenine modification or cytosine modification comprises methylation.

21. The method of claim 19, wherein the cytosine modification comprises 5-methylcytosine, 5-hydroxymethlcytosine, 5-formylcytosine, 5-carboxylcytosine, 5-glucosylhydroxylmethylcytosine or 3-methylcytosine.

22. The method of any one of claims 16-21, wherein the second modification-sensitive restriction enzyme comprises a restriction enzyme selected from the group consisting of AbaSI, FspEI, LpnPI, MspJI or McrBC.

23. The method of any one of claims 1-22, wherein the nucleic acids targeted for depletion comprise host nucleic acids and the nucleic acids of interest comprise non-host nucleic acids.

24. The method of claim 23, wherein the non-host comprises a bacterium, a fungus or a virus.

25. The method of claim 23, wherein the non-host comprises multiple species of organisms.

26. The method of claim 23, wherein the host is a mammal, a bird, a reptile or an insect.

27. The method of claim 26, wherein the mammal is a human.

28. The method of any one of claims 1-27, wherein the nucleic acids targeted for depletion comprise transcriptionally active sites and the nucleic acids of interest comprise repetitive sequences.

29. The method of any one of claims 1-28, wherein the adapter-ligated nucleic acids of interest and nucleic acids targeted for depletion range from 50-1000 bp.

30. The method of any one of claims 1-29, wherein the sample is any one of a biological sample, a clinical sample, a forensic sample or an environmental sample.

31. A method of enriching a sample for nucleic acids of interest comprising:
a. providing a sample comprising nucleic acids of interest and nucleic acids targeted for depletion, wherein at least a subset of the nucleic acids targeted for depletion comprise a plurality of recognition sites for a modification-sensitive restriction enzyme;
b. terminally dephosphorylating a plurality of the nucleic acids in the sample;
c. contacting the sample from (b) with the modification-sensitive restriction enzyme under conditions that allow for the cleavage of the modification-sensitive restriction sites in the nucleic acids in the sample, thereby generating nucleic acids with exposed terminal phosphates; and
d. contacting the sample with an exonuclease under conditions that allow for the successive removal of nucleotides from a phosphorylated end of a nucleic acid; thereby generating a sample enriched for nucleic acids of interest.

32. The method of claim 31, wherein both the nucleic acids of interest and the nucleic acids targeted for depletion comprise a plurality of recognition sites for the modification-sensitive restriction enzyme.

33. The method of claim 32, wherein the plurality of recognition sites in the nucleic acids targeted for depletion are modified more frequently than the plurality of recognition sites in the nucleic acids of interest.

34. The method of any one of claims 31-33, wherein the nucleic acids of interest comprise at least one DpnI recognition site, and wherein the method further comprises, prior to step (c), contacting the sample with DpnI and T4 polymerase thereby replacing methylated A and C nucleotides with unmethylated A and C nucleotides within or adjacent to the at least one DpnI recognition site.

35. The method of any one of claims 31-34, wherein the modification comprises adenine modification or cytosine modification.

36. The method of claim 35, wherein the adenine modification or cytosine modification comprises methylation.

37. The method of claim 35, wherein the cytosine modification comprises 5-methylcytosine, 5-hydroxymethlcytosine, 5-formylcytosine, 5-carboxylcytosine, 5-glucosylhydroxymethylcytosine or 3-methylcytosine.

38. The method of any one of claims 31-37, wherein the modification-sensitive restriction enzyme comprises a restriction enzyme selected from the group consisting of AbaSI, FspEI, LpnPI, MspJI or McrBC.

39. The method of any one of claims 31-34, wherein the modification comprises 5-hydroxymethylcytosine, the modification-sensitive restriction enzyme comprises AbaSI, and the method further comprises contacting the sample with T4 phage β-glucosyltransferase prior to step (c).

40. The method of any one of claims 31-34, wherein the modification comprises glucosylhydroxymethylcytosine, and the modification-sensitive restriction enzyme comprises AbaSI.

41. The method of any one of claims 31-34, wherein the modification comprises methylcytosine, and the modification-sensitive restriction enzyme comprises McrBC.

42. The method of any one of claims 31-41, further comprising:
e. contacting the sample from (d) with adapters under conditions that allow for the ligation of the adapters to a 5′ and 3′ end of a plurality of the nucleic acids of interest;
thereby generating a sample enriched for nucleic acids of interest that are adapter-ligated on their 5′ and 3′ ends.

43. The method of any one of claims 31-42, further comprising contacting the sample after step (d) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends.

44. The method of any one of claims 31-43, further comprising amplifying, sequencing or cloning the nucleic acids of interest that are adapter-ligated on their 5′ and 3′ ends using the adapters.

45. The method of any one of claims 31-44, wherein the nucleic acids targeted for depletion comprise host nucleic acids and the nucleic acids of interest comprise non-host nucleic acids.

46. The method of claim 45, wherein the non-host comprises a bacterium, a fungus or a virus.

47. The method of claim 45, wherein the host is a human.

48. The method of any one of claims 31-47, wherein the nucleic acids targeted for depletion comprise transcriptionally active sites and the nucleic acids of interest comprise repetitive sequences.

49. The method of any one of claims 31-48, wherein the adapter-ligated nucleic acids of interest and nucleic acids targeted for depletion range from 50-1000 bp.

50. The method of any one of claims 31-49, wherein the sample is any one of a biological sample, a clinical sample, a forensic sample or an environmental sample.

51. A method of enriching a sample for nucleic acids of interest comprising:
a. providing a sample comprising nucleic acids of interest and nucleic acids targeted for depletion, wherein at least a subset of the nucleic acids targeted for depletion comprise a plurality of recognition sites for a modification-sensitive restriction enzyme;
b. contacting the sample with adapters under conditions that allow for the ligation of the adapters to a 5′ and 3′ end of a plurality of the nucleic acids in the sample; and
c. contacting the sample from (b) with the modification-sensitive restriction enzyme under conditions that allow for the cleavage of the modification-sensitive restriction sites in the nucleic acids in the sample;
thereby generating a sample enriched for nucleic acids of interest that are adapter-ligated on their 5′ and 3′ ends.

52. The method of claim 51, wherein both the nucleic acids of interest and the nucleic acids targeted for depletion comprise a plurality of recognition sites for the modification-sensitive restriction enzyme.

53. The method of claim 51 or 52, wherein the plurality of recognition sites in the nucleic acids targeted for depletion are modified more frequently than the plurality of recognition sites in the nucleic acids of interest.

54. The method of any one of claims 51-53, wherein the nucleic acids of interest comprise at least one DpnI recognition site, and wherein the method further comprises, prior to step (c), contacting the sample with DpnI and T4 polymerase thereby replacing methylated A and C nucleotides with unmethylated A and C nucleotides within or adjacent to the at least one DpnI recognition site.

55. The method of any one of claims 51-54, wherein the modification comprises adenine modification or cytosine modification.

56. The method of claim 55, wherein the adenine modification or cytosine modification comprises methylation.

57. The method of claim 55, wherein the cytosine modification comprises 5-methylcytosine, 5-hydroxymethlcytosine, 5-formylcytosine, 5-carboxylcytosine, 5-glucosylhydroxymethylcytosine or 3-methylcytosine.

58. The method of any one of claims 51-57, wherein the modification-sensitive restriction enzyme comprises AbaSI, FspEI, LpnPI, MspJI or McrBC.

59. The method of any one of claims 51-53, wherein the modification comprises 5-hydroxymethylcytosine, the modification-sensitive restriction enzyme comprises AbaSI, and the method further comprises contacting the sample with T4 phage β-glucosyltransferase prior to (c).

60. The method of any one of claims 51-53, wherein the modification comprises glucosylhydroxymethylcytosine and the modification-sensitive restriction enzyme comprises AbaSI.

61. The method of any one of claims 51-53, wherein the modification comprises methylcytosine, and the modification-sensitive restriction enzyme comprises McrBC.

62. The method of any one of claims 51-61, further comprising contacting the sample after step (c) with a plurality of nucleic acid-guided nuclease-guide nucleic acid (gNA) complexes, wherein the gNAs are complementary to targeted sites in the nucleic acids targeted for depletion, thereby generating cut nucleic acids targeted for depletion that are adapter-ligated on one end and nucleic acids of interest that are adapter-ligated on both the 5′ and 3′ ends.

63. The method of any one of claims 51-62, further comprising amplifying, sequencing or cloning the nucleic acids of interest that are adapter-ligated on their 5′ and 3′ ends using the adapters.

64. The method of any one of claims 51-63, wherein the nucleic acids targeted for depletion comprise host nucleic acids and the nucleic acids of interest comprise non-host nucleic acids.

65. The method of claim 64, wherein the non-host comprises a bacterium, a fungus or a virus.

66. The method of claim 65, wherein the host is a human.

67. The method of any one of claims 51-66, wherein the nucleic acids targeted for depletion comprise transcriptionally active sites and the nucleic acids of interest comprise repetitive sequences.

68. The method of any one of claims 51-67, wherein the adapter-ligated nucleic acids of interest and nucleic acids targeted for depletion range from 50-1000 bp.

69. The method of any one of claims 51-68, wherein the sample is any one of a biological sample, a clinical sample, a forensic sample or an environmental sample.

70. A method of enriching a sample for nucleic acids of interest comprising:
a. providing a sample comprising nucleic acids of interest and nucleic acids targeted for depletion,
wherein at least a subset of the nucleic acids of interest or a subset of the nucleic acids targeted for depletion comprise a plurality of first recognition sites for a first modification-sensitive restriction enzyme, and
wherein activity of the first modification-sensitive restriction enzyme is blocked by modification of a nucleotide within or adjacent to its cognate recognition site;
b. terminally dephosphorylating a plurality of the nucleic acids in the sample;
c. contacting the sample from (b) with the first modification-sensitive restriction enzyme under conditions that allow for cleavage of at least some of the first modification-sensitive restriction sites in the nucleic acids in the sample; and
d. contacting the sample from (c) with adapters under conditions that allow for the ligation of the adapters to a 5′ and 3′ end of a plurality of the nucleic acids of interest;
thereby generating a sample enriched for nucleic acids of interest that are adapter-ligated on their 5′ and 3′ ends.
